(19) United States
(12) Patent Application Publication
(10) Pub. No.: US 2026/0165993 A1
(43) Pub. Date: Jun. 18, 2026

(54) METHODS OF REDUCING ALPHA-SYNUCLEIN AGGREGATES IN THE BRAIN WITH L-TYROSINE

(71) Applicant: BRAIN CHEMISTRY LABS, Jackson, WY (US)

(72) Inventors: Paul Alan COX, Jackson, WY (US); Sandra Anne BANACK, Jackson, WY (US)

(21) Appl. No.: 19/418,836

(22) Filed: Dec. 12, 2025

Related U.S. Application Data
(60) Provisional application No. 63/733,982, filed on Dec. 13, 2024.

(51) Int. Cl. A61K 31/198; A61P 25/16; A61P 25/28

(52) U.S. Cl. CPC A61K 31/198; A61P 25/16; A61P 25/28

(57) ABSTRACT

Disclosed herein are methods of reducing α-synuclein (α-syn) misfolds or aggregates in the brain, and specifically, to methods of treating neurodegenerative disorders caused by α-synuclein (α-syn) in the brain, also referred to as α-synucleinopathies, and to methods of hindering or preventing α-synucleinopathy misfolds, and/or or to methods of proactively treating individuals at risk of α-synucleinopathy misfolds and associated disease. These α-synucleinopathies refer primarily to Parkinson's disease (PD), dementia with Lewy Bodies (DLB), and/or Multiple system atrophy (MSA). One embodiment includes a method of administering an L-tyrosine compound or derivative thereof to a subject in need thereof, (optionally in combination with an L-serine compound), and reducing the density and/or amount of α-synuclein aggregates in the brain of a mammal, thereby treating a mammal diagnosed with or suffering from or at risk of suffering from one or more α-synucleinopathies.

### PRIORITY

This application claims priority to Provisional Patent Application Ser. No. 63/733,982, filed on Dec. 13, 2024, entitled: “METHODS OF REDUCING ALPHA-SYNUCLEIN AGGREGATES IN THE BRAIN WITH L-TYROSINE,” the disclosure of which is incorporated herein in its entirety.

### FIELD

The embodiments relate to methods of reducing α-synuclein (α-syn) aggregates in the brain, and specifically, to methods of treating neurodegenerative disorders caused by α-synuclein (α-syn) in the brain, also referred to as α-synucleinopathies. These refer primarily to Parkinson's disease (PD), dementia with Lewy Bodies (DLB), and Multiple system atrophy (MSA). One embodiment includes a method of administering an L-tyrosine compound or derivative thereof to a subject in need thereof, and reducing the density and/or amount of α-synuclein aggregates in the brain of a mammal, thereby treating a mammal diagnosed with or suffering from one or more α-synucleinopathies.

### BACKGROUND

Misfolded and aggregated α-synuclein (α-syn) initiates the formation of Lewy bodies (LBs), which may cause a loss of dopaminergic neurons, a hallmark of Parkinson's disease (PD). H. Fujiwara et al., alpha-Synuclein is phosphorylated in synucleinopathy lesions. Nat. Cell Biol. 4, 160-164 (2002); A. L. Mahul-Mellier et al., The process of Lewy body formation, rather than simply alpha-synuclein fibrillization, is one of the major drivers of neurodegeneration. Proc. Natl. Acad. Sci. U.S.A. 117, 4971-4982 (2020); C. W. Shults, Lewy bodies. Proc. Natl. Acad. Sci. U.S.A. 103, 1661-1668 (2006); and H. Braak et al., Staging of brain pathology related to sporadic Parkinson's disease. Neurobiol. Aging 24, 197-211 (2003). Likewise, misfolded α-syn can cause native α-syn proteins to misfold and aggregate (K. C. Luk et al., Pathological alpha-synuclein transmission initiates Parkinson-like neurodegeneration in nontransgenic mice. Science 338, 949-953 (2012)), leading to an increased density of synucleinopathies. E. Kovari, J. Horvath, C. Bouras, Neuropathology of Lewy body disorders. Brain Res. Bull. 80, 203-210 (2009).

Levodopa (L-DOPA) is the current standard of care for treatment of PD. After crossing the blood brain barrier (BBB), L-DOPA is decarboxylated to dopamine (E. Melamed, et al., Aromatic L-amino acid decarboxylase in rat corpus striatum . . . , Neurology, 31, 651-651 (1981)) thereby reducing motor symptoms. W. Birkmayer, et al., The effect of I-3,4-dihydroxyphenylalanine (=DOPA) on akinesia in parkinsonism. Parkinsonism Relat. Disord. 4, 59-60 (1998). However over time, administration of L-DOPA, even with increased dosages, has declining efficacy, does not slow disease progression, and can cause protein misfolding. S. W. Chan et al., L-DOPA is incorporated into brain proteins of patients treated for Parkinson's disease, inducing toxicity in human neuroblastoma cells in vitro. Exp. Neurol. 238, 29-37 (2012). Over an extended period, L-DOPA treatment for PD results in dyskinesia in some patients. Chan, et al., infra; S. Fahn et al., Levodopa and the progression of Parkinson's disease. N. Engl. J. Med. 351, 2498-2508 (2004); S. Giannopoulos, et al., L-DOPA causes mitochondrial dysfunction in vitro: A novel mechanism of L-DOPA toxicity uncovered. Int. J. Biochem. Cell Biol. 117, 105624 (2019); and G. Fabbrini, et al., Levodopa-induced dyskinesias. Mov. Disord., 22, 1379-1389 (2007).

The dietary amino acid L-tyrosine is a precursor in the biosynthesis of dopamine within the brain. It was evaluated in its relationship to PD beginning in the 1970s. Giannopoulos, et al., infra; J. H. Growdon, et al., Effects of oral L-tyrosine administration on CSF tyrosine and homovanillic acid levels in patients with Parkinson's disease. Life Sci. 30, 827-832 (1982); P. B. Molinoff, J. U. Axelrod, Biochemistry of catecholamines. Annu. Rev. Biochem. 40, 465-500 (1971); and A. Abbott, Levodopa: the story so far. Nature 466, S6-7 (2010). Since then, it has been found that reduced L-tyrosine intake is correlated with decreased dopamine concentrations (A. J. Montgomery, et al., Reduction of brain dopamine concentration with dietary tyrosine plus phenylalanine depletion: an [11C]raclopride PET study. Am. J. Psychiatry 160, 1887-1889 (2003)) while higher levels of L-tyrosine correlate with better cognition in patients with Lewy body dementia (LBD). A. McCann et al., Serum tyrosine is associated with better cognition in Lewy body dementia. Brain Res 1765, 147481 (2021). Exposures to acute neurotoxins can be lethal to dopaminergic neurons. V. Jackson-Lewis, et al., Protocol for the MPTP mouse model of Parkinson's disease. Nat. Protoc. 2, 141-151 (2007); N. W. Kowall et al., MPTP induces alpha-synuclein aggregation in the substantia nigra of baboons. Neuroreport 11, 211-213 (2000); and A. L. McCormack et al., Pathologic modifications of alpha-synuclein in 1-methyl-4-phenyl-1,2,3,6-tetrahydropyridine (MPTP)-treated squirrel monkeys. J. Neuropathol. Exp. Neurol. 67, 793-802 (2008). Chronic, rather than acute, dietary exposure to the cyanotoxin β-N-methylamino-L-alanine (BMAA), an environmental toxin linked to parkinsonism, triggers diverse proteinopathies in the brain. P. A. Cox, et al., Dietary exposure to an environmental toxin triggers neurofibrillary tangles and amyloid deposits in the brain. Proc. Biol. Sci. 283 (2016); S. A. Banack, et al., Creating a Simian Model of Guam ALS/PDC Which Reflects Chamorro Lifetime BMAA Exposures. Neurotox. Res. 33, 24-32 (2018).

### SUMMARY OF THE EMBODIMENTS

The embodiments herein are premised in part on the discovery that L-tyrosine, or precursors, derivatives, or conjugates thereof can protect against the formation of misfolded and aggregated α-syn in a mammalian brain. In accordance with the embodiments, there are provided methods and uses for L-tyrosine, or a precursor, derivative or conjugate of L-tyrosine in: preventing, inhibiting or reducing misfoldings and aggregates of α-syn in a mammalian brain. In one embodiment, a method or use includes contacting a cell with L-tyrosine, or a precursor, derivative or conjugate of L-tyrosine, and optionally L-serine, or a precursor, derivative or conjugate of L-serine, in amounts sufficient to prevent, inhibit or reduce misfoldings and aggregates of α-syn. In another embodiment, a method or use includes administering a composition comprising L-tyrosine, or a precursor, derivative or conjugate of L-tyrosine, and optionally L-serine, or a precursor, derivative or conjugate of L-serine, in amounts sufficient to prevent, inhibit or reduce misfolding or aggregation of α-syn in a mammalian brain, to thereby treat the mammal suffering from α-synucleinopathies.

In an embodiment, the α-synucleinopathies are selected from one or more of Parkinson's disease (PD), dementia with Lewy Bodies (DLB), and/or Multiple system atrophy (MSA), and more particularly PD. Other embodiments include, for example, methods and uses for L-tyrosine, or a precursor, derivative or conjugate of L-tyrosine, in reducing or decreasing risk of a neurological disease or disorder caused or characterized by misfolding or aggregation of α-syn; stabilizing, or reducing or inhibiting progression of, a neurological disease or disorder caused or characterized by misfolding or aggregation of α-syn; and treating a neurological disease or disorder caused or characterized by misfolding or aggregation of α-syn. In one embodiment, a method or use includes administering to the subject L-tyrosine, or a precursor, derivative or conjugate of L-tyrosine, and optionally L-serine, or a salt, metabolic precursor, derivative or conjugate of L-serine, in amounts sufficient to reduce or decrease risk of the neurological disease or disorder caused or characterized by misfolding or aggregation of α-syn. In another embodiment, a method or use includes administering to the subject L-tyrosine, or a precursor, derivative or conjugate of L-tyrosine, and optionally L-serine, or a salt, metabolic precursor, derivative or conjugate of L-serine, in amounts sufficient to stabilize, or reduce or inhibit progression of, a neurological disease or disorder a caused or characterized by misfolding or aggregation of α-syn. In a further embodiment, a method or use includes administering to the subject L-tyrosine, or a precursor, derivative or conjugate of L-tyrosine, and optionally L-serine, or a salt, metabolic precursor, derivative or conjugate of L-serine, in amounts sufficient to treat the neurological disease or disorder caused or characterized by misfolding or aggregation of α-syn.

Precursors, derivatives and conjugates of L-tyrosine include an L-tyrosine polymer (polytyrosine), a salt of L-tyrosine, an alkylated L-tyrosine or an L-tyrosine lipid. Precursors, derivatives and conjugates of L-tyrosine also include salts, such as a sodium salt, a potassium salt, a calcium salt, a magnesium salt, a zinc salt, or an ammonium salt of L-tyrosine. Precursors, derivatives and conjugates of L-tyrosine further include an alkylated L-tyrosine, such as L-tyrosine with an alkyl group, e.g., an alkyl comprising 1-20 carbon atoms. Precursors, derivatives and conjugates of L-tyrosine moreover include an L-tyrosine ester, an L-tyrosine di-ester, a phosphate ester of L-tyrosine, a sulfate or sulfonate ester of L-tyrosine, a pegylated L-tyrosine, a lipidated L-tyrosine or an L-tyrosine with one or more polyethylene glycol (PEG) moieties. A non-limiting example of a precursor of L-tyrosine is L-phosphoserine.

Non-limiting examples of a salt of L-serine include a sodium salt, potassium salt, calcium salt, magnesium salt, zinc salt, ammonium salt; inorganic salts such as, hydrogen chloride, sodium chloride, potassium chloride, calcium chloride, sodium phosphate, potassium phosphate, and sodium hydrogen carbonate; organic salts such as, sodium citrate, citrate, acetate, and the like. In certain embodiments, a composition comprises L-serine as an alkylated L-serine, such as L-serine with an alkyl group, or e.g., an alkyl comprising 1-20 carbon atoms. In certain embodiments, a derivative of L-serine includes an L-serine ester, an L-serine di-ester, a phosphate ester of L-serine, or a sulfate or sulfonate ester of L-serine. Non-limiting examples of a conjugate of L-serine includes a pegylated L-serine (e.g., an L-serine comprising one or more polyethylene glycol (PEG) moieties), and a lipidated L-serine. Non-limiting example of a precursor of L-serine include L-phosphoserine.

Non-limiting examples of a precursor of L-serine include a pro-form of L-serine that is broken down into L-serine monomers by the digestive system of a subject. In some embodiments, L-serine or a conjugate thereof consists of a slow-release version. In some embodiments a derivative of L-serine is conjugated to a different molecule forming a prodrug from which L-serine is released after crossing the blood/brain barrier.

Precursors, derivatives and conjugates of L-tyrosine and/or L-serine can be formulated into a composition or formulation, such as a pharmaceutical composition or formulation, including for example, tablets, capsules, gummies, jelly beans, and the like. Precursors, derivatives and conjugates of L-tyrosine and/or L-serine can also be included in liposomes or micelles. Such compositions and formulations, including pharmaceutical compositions, formulations, liposomes and micelles, include those suitable for administration or delivery by any route, such as orally, by injection, by infusion, by intubation, via catheter, intraspinally, or intracranially.

Methods and uses of the embodiments include those that provide a subjective or objective improvement in any symptom of a neurological disease or disorder, as set forth herein, or that is known to one of skill in the art. In particular embodiments, a method or use prevents, reduces or inhibits onset, severity, frequency, or duration of one or more symptoms of a neurological disease or disorder.

Methods and uses of the invention include administration, delivery or contact of a subject, or any tissue organ or cell of a subject, with any compatible means for delivery or contact of L-tyrosine, precursor, derivative or conjugate of L-tyrosine and/or L-serine, or a salt, metabolic precursor, derivative or conjugate of L-serine. In particular embodiments, L-tyrosine, precursor, derivative or conjugate of L-tyrosine, and/or L-serine, or a salt, metabolic precursor, derivative or conjugate of L-serine, are administered orally by injection, by infusion, by intubation, via catheter or intracranially to a subject in a method or use. In more particular aspects, L-tyrosine, precursor, derivative or conjugate of L-tyrosine, and/or L-serine, or a salt, metabolic precursor, derivative or conjugate of L-serine are administered at least once daily for at least one week to a subject; at least two, three, four, five, six, seven, 8, 9, 10, 11 or 12 weeks to a subject or at least one, two, three, four, five, six, seven, 8, 9, 10, 11 or 12 months to a subject. In embodiments, the compositions can be administered in increasing amounts, for example, 10 dosages a day for two weeks, 20 dosages a day for an additional two weeks, and 30 dosages a day thereafter to obtain the final amounts of L-tyrosine, precursor, derivative or conjugate of L-tyrosine, and/or L-serine, or a salt, metabolic precursor, derivative or conjugate of L-serine administered per day.

Methods and uses of the invention include doses of L-tyrosine, precursor, derivative or conjugate of L-tyrosine, optionally intended to achieve a desired effect. In particular embodiments, L-tyrosine, precursor, derivative or conjugate of L-tyrosine, is administered at a dose of about 1-5 g/day, 5-10 g/day, 10-15 g/day, 15-20 g/day, 20-25 g/day, 25-30 g/day, 30-40 g/day, 40-50 g/day 50-75 g/day or 75-100 g/day to a subject; or is administered at a dose of about 1-10 mg/kg body weight, 10-25 mg/kg body weight, 25-50 mg/kg body weight, 50-100 mg/kg body weight, 100-250 mg/kg body weight, 250-500 mg/kg body weight, 500-750 mg/kg body weight, or 750-1,000 mg/kg body weight to a subject.

Methods and uses of the invention include doses of L-serine, or a salt, metabolic precursor, derivative or conjugate of L-serine, optionally intended to achieve a desired effect. In particular embodiments, L-serine, or a salt, metabolic precursor, derivative or conjugate of L-serine, is administered at a dose of about 0.5-10 g/day, 1-7.5 g/day, 2.5-6 g/day, or 5 g/day to a subject; or is administered at a dose of about 0.5-10 mg/kg body weight, 10-25 mg/kg body weight, 25-50 mg/kg body weight, 50-100 mg/kg body weight, 100-250 mg/kg body weight, or 250-500 mg/kg to a subject.

### DESCRIPTION OF THE DRAWINGS

FIG. 1 illustrates how supplementation with L-tyrosine protects and improves executive function. (a) Two independent experiments were performed to assess the effect of the BMAA toxin on executive function and evaluate of whether supplementation with neuroprotective amino acids L-serine (SER) and L-tyrosine (TYR) could protect against the toxin; (x)=not tested. (b) Marmosets were challenged with the detour reaching task (DRT) which involves training of the animal to obtain a food prize with a direct reach followed by trying to obtain the same prize using a detour reach. (c) In the first experiment, control (CTL, N=2) animals performed better than those fed BMAA (n=4 marmosets/2 males, 2 females, average shown). (d) L-tyrosine reduced the number of sessions require to reach criterion in the DRT, while Marmosets fed BMAA and BMAA+L-Serine required the most sessions (N=4 marmosets/2 males, 2 females, average shown) Numbers of sessions needed to reach criterion (p=0.011). The interaction between group and sex also significant (p=0.021). In FIG. 1, panels a & b were created using BioRender.com.

FIG. 2 illustrates how β-N-methylamino-L-alanine increases the expression of phosphorylated serine 129-synuclein protein. Representative images of brain sections immunostained with phosphorylated serine 129 α-synuclein (pS129 α-syn) from the first experiment. Chronic dietary exposure to β-N-methylamino-L-alanine (BMAA) increased the expression of pS129 α-syn in the entorhinal cortex (Ent) by 40% (a-c), the amygdala (Amg) by 190% (d-f), the hippocampus (Hip) by 120% (g-i) and the frontal cortex (Fc) by 40% (j-1) (p<0.0001; n=120 microscopic scan fields per treatment, Wilcoxon test). White arrows depict large aggregates in the hippocampal CA3 field (h). While other lesions ranged from diffused and focal neuronal staining in the motor cortex and brainstem (m, n) and Lewy body-like structures in the cerebral cortex and mid brain (o, p). Digital scan magnification: 5× (a, b); 10× (d, e); 20× (g, h, j, k); 60× (m-p).

FIG. 3 illustrates how L-tyrosine reduces α-synuclein aggregates and pathological tau deposition. (a) Representative images of the frontal cortex (Fc), a region involved in the execution of the detour reaching task, immunostained with phosphorylated serine 129 α-synuclein (pS129 α-syn) with no counterstain from animals in the second experiment. (b, c) Compared to BMAA treated marmosets, co-supplementation with L-tyrosine (TYR), reduced the expression of total pS129 α-syn and α-syn aggregates counts up to 98% (p<0.0001, N=40 normalized microscopic scan fields/treatment group). (d) TYR effects were similar on expression of Tau-AT8, a pathological form of tau protein that makes up neurofibrillary lesions, were the expression was reduced 94% (p<0.0001, N=40 normalized microscopic scan fields/treatment group). (e) In BMAA treated animals, ranked Tau-AT8 expression positively correlated with pS129 α-syn aggregates (p<0.0001, N=40 scan fields/treatment group). Digital scan magnification 10×.

FIG. 4 shows a phosphorylated α-synuclein expression analysis. (a-f) In order to validate the α-synuclein quantitative analysis, a series of staining positive control slides from adult A₅₃T mice brains stained with pS129 α-syn provided by NeuroScience Associates (NSA, Knoxville Tennessee, USA) were used. Using Objective View, a 20× field (dotted square) was exported from all 3 brains to FIJI software to generate a 1216×702 pixels image. The FIJI TIFF file was then imported into QuPath were a 200-pixel grid was applied to the image. (g) QuPath positive cell feature was used to measure 10 microscopic scan fields (for 200 μm²) on each image. The QuPath positive cell analysis feature allows for the selection of cell bodies over background staining (blue) and highlighting cells that are positive or overall a threshold of intensity (red). The following graphs highlighted data analyzed from 10 scan fields: (h) the number of positive cells for 200 μm², (i) the percentage of positive cells, or (j) the mean optical density of positive cells over baseline threshold (dotted line). (*, p<0.05, ****, p<0.0001; ANOVA). Digital scan magnification: 1.25× (a-c); 5× (d); 20× (e-g).

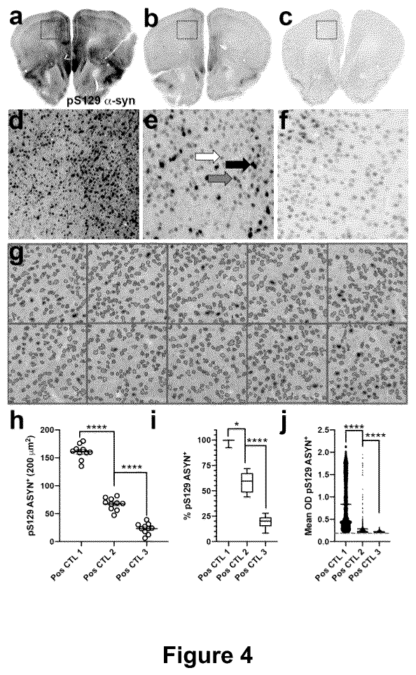

FIG. 5 shows paired helical filament tau protein accumulation in BMAA exposed marmosets. Chronic exposure to BMAA in Experiment 1 increased the severity of paired helical filament (PHF) tau proteins in the marmoset brain. (a, b) Coronal section at the level of the amygdala of a marmoset fed BMAA for 140 days and a control. (c) Correlation of rank PHF-tau and pS129 α-syn expression across all four brain regions analysed (r=0.9870, Spearman Rank). Panels (d-e) are magnified images from a and b. BMAA increased Tau AT8 aggregation in the amygdala (d-f), the entorhinal cortex (g-h), hippocampus (j-i) and the frontal cortex (m-o) (n=120 scan fields/treatment group; p<0.0001; Friedman test). Abbreviations: Amg, amygdala, Ce, central nucleus of the amygdala, Ent, entorhinal cortex, opt, optic tract, Pal, paralaminar nucleus of the amygdala, Te, temporal region, and rf, rhinal fissure. Digital scan magnification: 1.25× (a, b); 20× (d, e); 10× (g, h, j, k).

FIG. 6 shows a pS129 α-synuclein aggregate analysis. The inventors observed two major types of α-syn staining patterns: diffused intracellular and small punctate. The latter being more associated with α-syn protein aggregation. Using QuPath software, we applied a modified threshold to capture, delineate, quantify, and summarize these two staining patterns. (a) In the Total analysis, we quantified all pS129 α-syn positive counts above threshold (see FIG. S1). The hippocampus is shown here as an example. (b) Using a modified threshold, we were able to select only small punctate and dense pS129 α-syn staining patterns, excluding those above a designated size and morphology. This analysis type was term Aggregate analysis. Using this analysis type, only aggregates are selected in the same CA3 field. (c) Spinal cord anterior horn analyzed with the Total analysis or “all inclusive” threshold. Positive detections are shown in red and negative detections (below threshold) are shown in blue. (d) shows the identical spinal cord analysis fields in (c), but with the modified “Aggregate analysis” threshold applied. The larger and diffused intracellular staining patterns have been excluded from the analysis. Digital scan magnification: (a, b) 12×; (c, d) 20×.

FIG. 7 illustrates how L-Tyrosine reduces pS129 α-synuclein aggregates in the spinal cord. (a-d) Representative low power images of marmoset spinal cords from the second experiment: BMAA, BMAA+L-serine (SER), BMAA+L-tyrosine (TYR), and control. Three distinct anatomical regions of the spinal cord gray matter are presented in higher power images below: 1: posterior horn (e-h), 2: lateral horn (i-I), and 3: anterior horn (m-p). BMAA increased pS129 α-syn aggregates in the anterior, lateral and posterior horns. Dietary supplementation with L-tyrosine reduced the number of aggregates in the spinal cord. Digital scan magnification: 2.5× (a-d); 20× (e-p).

FIG. 8 shows how BMAA induces confirmation specific α-synuclein aggregates in the frontal cortex. To validate alpha synucleinopathy in our marmoset model, we applied a conformation-specific anti-α-synuclein aggregate antibody [MJFR-14-6-4-2] to the frontal cortex (Fc) of marmosets from the second experiment. (a) Shows a section midbrain from postmortem human brain with end stage Parkinson disease. The MJFR-14-6-4-2 antibody labels Lewy bodies (white arrows) and a Lewy neurite. (b-e) Frontal cortex sections immunostained with MJFR-14. A cluster of α-syn aggregates were located in lateral Fc of a BMAA treated marmosets and then the same anatomical location was evaluated in control, L-serine and L-serine treatments on the same MultiBraintm Block slide. «-syn aggerates average 2 microns in diameter (N=17 aggregates) using CONCENTRIQ software measuring tool (Proscia, Philaphedia, PA, USA). Digital scan magnification 20× (a) 36× (b-e).

### DETAILED DESCRIPTION

In accordance with the embodiments, there are provided methods and uses for L-tyrosine, or a precursor, derivative or conjugate of L-tyrosine, and/or L-serine, or a salt, metabolic precursor, derivative or conjugate of L-serine in: preventing, inhibiting or reducing misfoldings and aggregates of α-syn in a mammalian brain. In one embodiment, a method or use includes contacting a cell with L-tyrosine, or a precursor, derivative or conjugate of L-tyrosine, and/or L-serine, or a salt, metabolic precursor, derivative or conjugate of L-serine in amounts sufficient to prevent, inhibit or reduce misfoldings and aggregates of α-syn. In another embodiment, a method or use includes administering a composition comprising L-tyrosine, or a precursor, derivative or conjugate of L-tyrosine and/or L-serine, or a salt, metabolic precursor, derivative or conjugate of L-serine in amounts sufficient to prevent, inhibit or reduce misfolding or aggregation of α-syn in a mammalian brain, to thereby treat the mammal suffering from α-synucleinopathies.

In an embodiment, the α-synucleinopathies are selected from one or more of Parkinson's disease (PD), dementia with Lewy Bodies (DLB), and/or Multiple system atrophy (MSA), and more particularly PD.

Levodopa (L-DOPA) improves motor symptoms in Parkinson's disease (PD), but prolonged treatment does not slow aggregation of misfolded alpha-synuclein and can induce dyskinesia in some patients. The amino acid L-tyrosine is converted to dopamine in the brain and has been found to be safe in clinical trials. The inventors induced α-synuclein aggregates and Lewy bodies in marmosets (Callithrix jacchus) through chronic dietary exposure to an environmental toxin associated with parkinsonism. When challenged with the detour reach task, the inventors discovered that their ability to solve a simple puzzle for a food reward was impaired. Microscopic evaluation of the dorsal lateral frontal cortex, a brain region associated with the detour reach task, demonstrated on average a three-fold increase in α-synuclein aggregates in the brain. However, marmosets which were co-administered L-tyrosine with the toxin experienced significantly lower (97% decrease) density of neuropathology and improved performance in puzzle solving (76% increase). L-tyrosine protective effects against α-synuclein aggregation and cognition were observed in both female and male marmosets. These results suggest that L-tyrosine should be evaluated as a potential therapeutic or adjunct in the treatment of α-synucleinopathies. PD is the second most common neurodegenerative disease with a 20-year prodromal period.

The inventors sought to determine if chronic dietary exposure in marmosets could cause accumulation of aggregated α-syn and if co-administration with L-tyrosine could protect against the formation of misfolded and aggregated α-syn. In a first experiment, chronic dietary exposure to BMAA and its results on cognitive function was assessed using the detour reach task (DRT) behavioral paradigm. C. Kabadayi, et al., The detour paradigm in animal cognition. Anim. Cogn. 21, 21-35 (2018). Following chronic toxin exposure, expression of phosphorylated serine 129 α-syn (pS129 α-syn), a form of α-syn that increases from about 4% under normal physiological conditions to 90% in PD was evaluated. S. S. Ghanem et al., α-Synuclein phosphorylation at serine 129 occurs after initial protein deposition and inhibits seeded fibril formation and toxicity. Proc. Natl. Acad. Sci. U.S.A. 119, e2109617119 (2022). In addition, toxin treated animals as well as their pS129 α-syn expression was assessed in the frontal cortex, a region associated with the execution of the DRT behavioral task, as well as several brain regions previously shown to be vulnerable to BMAA neurotoxicity and related to cognitive decline (i.e., the amygdala, hippocampus and entorhinal cortex).

In a second experiment, L-tyrosine and L-serine arms-both co-administered with the BMAA toxin was tested. The dietary amino acid L-serine had been previously shown to reduce BMAA-induced neurotoxicity in non-human primates. D. A. Davis et al., L-serine reduces spinal cord pathology in a vervet model of preclinical ALS/MND. J. Neuropath. Exp. Neurol. 79, 393-406 (2020). The inventors then expanded their analysis to sixteen brain regions associated with several α-synucleinopathies. Braak, et al., infra, and I. Alafuzoff et al., Staging/typing of Lewy body related alpha-synuclein pathology: a study of the BrainNet Europe Consortium. Acta Neuropathol. 117, 635-652 (2009). After chronic dietary exposure to the toxin and behavioral testing, quantitative immunohistochemistry was performed to evaluate phosphorylated α-syn expression and misfolded α-syn aggregate formation in the brain and spinal cord.

Although L-tyrosine is a precursor in the biosynthesis of dopamine in the brain, as early as the 1970s it was predicted that “Parkinsonism cannot be effectively treated by feeding an excess of L-tyrosine.” D. B. Calne, M. Sandler, L-Dopa and parkinsonism. Nature 226, 21-24 (1970). The in vivo data created by the inventors suggest that these theoretical predictions may not be correct. Preliminary studies indicate L-tyrosine is well tolerated in patients using Levodopa, it increases dopamine in the CNS, and facilitates cognitive flexibility. J. DiFrancisco-Donoghue, et al., Effects of tyrosine on Parkinson's disease: a randomized, double-blind, placebo-controlled trial. Mov. Disord. Clin. Pract. 1, 348-353 (2014); L. Steenbergen, et al., Tyrosine promotes cognitive flexibility: evidence from proactive vs. reactive control during task switching performance. Neuropsychologia 69, 50-55 (2015); and M. Bloemendaal et al., Neuro-cognitive effects of acute tyrosine administration on reactive and proactive response inhibition in healthy older adults. Eneuro 5, (2018).

Potentially, L-tyrosine could be evaluated for use for the prodromal period in PD, which can start as early as 20 years before a clinical diagnosis as well as in advanced stages of PD to slow the conversion of PD patients to mild cognitive impairment (PD-MCI) and their subsequent conversion to dementia (PD-D). C. Klein, A. Westenberger, Genetics of Parkinson's disease. Cold Spring Harb. Perspect. Med. 2, a008888 (2012); R. Savica, et al., When does Parkinson disease start? Arch. Neurol. 67, 798-801 (2010); and D. Saredakis, et al., Conversion to MCI and dementia in Parkinson's disease: a systematic review and meta-analysis. Parkinsonism Relat. Disord. 65, 20-31 (2019). For advanced patients with PD-MCI and PD-D, few pharmacological treatments are available.

The term “ameliorate” means a detectable or measurable objective or subjective improvement in a subject's condition. A detectable or measurable improvement includes a subjective or objective reduction in the severity or frequency of one or more symptoms caused by or associated with the disorder or disease, or an improvement in the underlying causes of the disorder or disease, or a reversal of the disorder or disease.

Stabilizing a disease or disorder is also a successful treatment outcome. A successful treatment can reduce or prevent the severity or frequency of one or more symptoms of the disease or disorder, inhibit progression or worsening of the disease or disorder, and in some instances, reverse the disease or disorder. Accordingly, in the case of a neurological disease or disorder, for example, treatment can lead to an improvement of one or more symptoms of the neurological disease or disorder, stabilizing one or more symptoms of the neurological disease or disorder, or a reversal of the neurological disease or disorder.

Non-limiting symptoms of neurological disorders and diseases include, but are not limited to: motor (e.g., coordination) or cognitive deficiency; fatigue; tremors; ataxia; slurred, thick or irregular speech; muscle cramps, twitching, atrophy or weakness (e.g., hands, arms, legs, swallowing, breathing, speech muscles); shortness of breath; eating, breathing or swallowing difficulty. Non-limiting symptoms of neurological disorders and diseases also include, but are not limited to: short term or long term memory loss; difficulty concentrating or completing familiar or routine tasks; space and time confusion; vision, color or sign recognition loss; depth perception loss, speaking or writing difficulty; loss of judgment; vocabulary loss; moodiness; irritability; aggression; paranoia; delusions; and withdrawal from social engagement. Non-limiting symptoms of neurological disorders and diseases further include, but are not limited to: tremor; stiffness or rigidity; loss of fine or gross motor control; slowing of movement; impaired balance; body instability; posture or gait abnormality; and shuffling walk. Non-limiting symptoms of neurological disorders and diseases additionally include, but are not limited to: reduced coordination; physical instability; unsteady gait; motor dysfunction; jerky body movement (chorea); slowed saccadic eye movement; body rigidity; seizure; difficulty chewing, eating, swallowing or speaking; deterioration in cognition/mental capabilities including dementia; short and/or long term memory loss; sleep, behavioral and psychiatric abnormalities (e.g., anxiety, depression, loss of emotional affect, aggression, compulsory behavior). Non-limiting symptoms of neurological disorders and diseases moreover include, but are not limited to: difficulty in speech and thinking; behavioral changes; impaired regulation of social conduct (e.g. breaches of etiquette, tactlessness, dis-inhibition, criminal behavior); passivity; lethargy; social withdrawal; inertia; over-activity; pacing; wandering. Non-limiting symptoms of neurological disorders and diseases still further include, but are not limited to: loss of balance; lunging forward when mobilizing; fast walking; imbalance, e.g., bumping into objects or people; falls; changes in personality; slowing of movement; dementia (including loss of inhibition and ability to organize information); slurred speech; difficult swallowing; opthalmoparesis or impaired eye movement (particularly in the vertical direction which accounts for some of the falls experienced by patients); impaired eyelid function; facial muscle contracture; Neck dystonia or backward tilt of the head with stiffening of neck muscles; sleep disruption; urinary/bowel incontinence; and parkinsonism. Methods and uses, such as treatment in accordance with the embodiments, include treatment that improves or ameliorates any one of the foregoing symptoms, to any degree or duration of time.

Methods and uses, such as treatment in accordance with the embodiments, also include affecting the underlying causes of the disease or disorder thereof. Thus, in the case of a neurological disease or disorder, for example, stabilizing or decreasing worsening of the condition, for example, by objective and subjective measures of clinical severity of the neurological disorder is considered a successful treatment outcome.

In a method or use of the invention in which a therapeutic benefit or improvement is a desired outcome, L-tyrosine, or a precursor, derivative or conjugate of L-tyrosine, and/or L-serine, or a salt, metabolic precursor, derivative or conjugate of L-serine can be administered in sufficient or effective amounts to a subject in need thereof. An “amount sufficient” or “amount effective” refers to an amount that is calculated or likely to provide, in single or multiple doses, alone or in combination with one or more other compositions (therapeutic agents such as a chemotherapeutic or immune stimulating drug), treatments, or therapeutic protocols, regimens or agents, a detectable response of any duration of time (long or short term), a desired outcome in or a benefit to a subject of any measurable or detectable degree or for any duration of time (e.g., for hours, days, months, years, or cured). The dose or “sufficient amount” or “effective amount” for treatment (e.g., to provide a therapeutic benefit or improvement) typically is effective to ameliorate a disorder or disease, or one, multiple or all adverse symptoms, consequences or complications of the disorder or disease, to a measurable extent, although reducing or inhibiting a progression or worsening (e.g., stabilizing) of the disorder or disease or a symptom, is considered a satisfactory outcome.

Treatment can therefore result in inhibiting, reducing or preventing a disorder or disease, or an associated symptom or consequence, or underlying cause; inhibiting, reducing or preventing a progression or worsening of a disorder or disease, symptom or consequence, or underlying cause; or further deterioration or occurrence of one or more additional symptoms of the disorder or disease, or symptom. Thus, a successful treatment outcome leads to a “therapeutic effect,” or “benefit” or inhibiting, reducing or preventing the occurrence, frequency, severity, progression, or duration of one or more symptoms or underlying causes or consequences of a disorder or disease, in the subject. Treatment methods and uses affecting one or more underlying causes of the disorder or disease or a symptom are therefore considered beneficial. Stabilizing or inhibiting or reducing progression or worsening of a disorder or disease is also a successful treatment outcome.

A therapeutic benefit or improvement therefore need not be complete ablation of any one, most or all symptoms, complications, consequences or underlying causes associated with the disorder or disease. Thus, a satisfactory endpoint is achieved even where only an incremental improvement in a subject's condition is achieved, such as a partial reduction in the occurrence, frequency, severity, progression, or duration, or inhibition or reversal, of one or more associated adverse symptoms or complications or consequences or underlying causes, worsening or progression (e.g., stabilizing one or more symptoms or complications of the disease or disorder), of one or more of the physiological, biochemical or cellular manifestations or characteristics of the disorder or disease, over a short or long duration of time (minutes, hours, days, weeks, months, etc.).

A therapeutic benefit or treatment efficacy can be observed or measured by improvement in any one or more of the symptoms that characterize or are associated with the neurological disorder or disease as set forth herein. Particular non-limiting examples of therapeutic benefit or improvement for a neurological disorder or disease include, but is not limited to any of the following symptoms (e.g., a reduction or decrease of an adverse symptom or progression of an adverse symptom): motor (e.g., coordination) or cognitive deficiency; fatigue; tremors; ataxia; slurred, thick or irregular speech; muscle cramps, twitching, atrophy or weakness (e.g., hands, arms, legs, swallowing, breathing, speech muscles); shortness of breath; eating, breathing or swallowing difficulty; short term or long term memory loss; difficulty concentrating or completing familiar or routine tasks; space and time confusion; vision, color or sign recognition loss; depth perception loss, speaking or writing difficulty; loss of judgment; vocabulary loss; moodiness; irritability; aggression; paranoia; delusions; withdrawal from social engagement; tremor; stiffness or rigidity; loss of fine or gross motor control; slowing of movement; impaired balance; body instability; posture or gait abnormality; shuffling walk; reduced coordination; physical instability; unsteady gait; motor dysfunction; jerky body movement (chorea); slowed saccadic eye movement; body rigidity; seizure; difficulty chewing, eating, swallowing or speaking; deterioration in cognition/mental capabilities including dementia; short and/or long term memory loss; sleep, behavioral and psychiatric abnormalities (e.g., anxiety, depression, loss of emotional affect, aggression, compulsory behavior); difficulty in speech and thinking; behavioral changes; impaired regulation of social conduct (e.g. breaches of etiquette, tactlessness, dis-inhibition, criminal behavior); passivity; lethargy; social withdrawal; inertia; over-activity; pacing; wandering; loss of balance; lunging forward when mobilizing; fast walking; imbalance, e.g., bumping into objects or people; falls; changes in personality; slowing of movement; dementia (including loss of inhibition and ability to organize information); slurred speech; difficult swallowing; opthalmoparesis or impaired eye movement (particularly in the vertical direction which accounts for some of the falls experienced by patients); impaired eyelid function; facial muscle contracture; neck dystonia or backward tilt of the head with stiffening of neck muscles; sleep disruption; urinary/bowel incontinence; and parkinsonism. Methods and uses, such as treatment in accordance with the invention, include treatment that improves or ameliorates any one of the foregoing symptoms, to any extent or period of time.

Additional examples include measurement of various functions compared to established criteria. For example, an assessment of intellectual (cognitive) functioning such as memory testing or physical functioning can further characterize state of a disease. A therapeutic benefit or treatment efficacy of a neurological disorder or disease (or severity of a neurological disorder or disease) can also be ascertained or measured by mechanical means or instrumentation. In particular, positron electron tomography (PET) scan of the brain of a person with a neurological disorder or disease can show the degree to which there is a loss of function. Imaging such as computed tomography (CT) or magnetic resonance imaging (MRI), or with single positron emission computed tomography (SPECT) or positron emission tomography (PET) can also be used to identify cerebral pathology or subtypes of dementia—in order to ascertain therapeutic benefit or treatment efficacy of a neurological disorder or disease, or severity of a neurological disorder or disease. These techniques therefore can be correlated with alpha-synuclein misfolds that are associated with the neurological disorder or disease.

For Parkinson's Disease, diagnosis of onset can be made following the appearance of physical and/or psychological symptoms specific to the disease, as set for the herein, for example. Characteristic features include tremor; body stiffness or rigidity; loss of fine or gross motor control; slowing of movement; impaired balance; body instability; posture or gait abnormality; and shuffling walk, reduced coordination; physical instability; unsteady gait; motor dysfunction; jerky body movement (chorea); slowed saccadic eye movement; seizure; difficulty chewing, eating, swallowing or speaking; deterioration in cognition/mental capabilities including dementia; short and/or long term memory loss; sleep, and behavioral and psychiatric abnormalities (e.g., anxiety, depression, loss of emotional affect, aggression, compulsory behavior).

An amount sufficient or an amount effective can but need not be provided in a single administration and, can but need not be, administered alone or in combination with another composition, treatment, protocol or therapeutic regimen. For example, the amount may be proportionally increased as indicated by the need of the subject, status of the disorder or disease treated or the side effects of treatment. In addition, an amount sufficient or an amount effective need not be sufficient or effective if given in single or multiple doses without a second composition, treatment, protocol or therapeutic regimen, since additional doses, amounts or duration above and beyond such doses, or additional compositions, treatments, protocols or therapeutic regimens may be included in order to be considered effective or sufficient in a given subject.

Amounts considered sufficient also include amounts that result in a reduction of the use of another treatment, therapeutic regimen or protocol. For example, a sufficient or effective amount of L-tyrosine, or a precursor, derivative or conjugate of L-tyrosine, and/or L-serine, or a salt, metabolic precursor, derivative or conjugate of L-serine is considered as having a therapeutic effect if administration results in less amount or frequency of a drug, or another therapy or protocol, being required to treat a neurological disorder or disease.

An amount sufficient or an amount effective need not be effective in each and every subject treated, prophylactically or therapeutically, in a particular subject, or a majority of treated subjects in a given group or population. As is typical for treatment or therapeutic methods, some subjects will exhibit greater or less response to a given treatment, therapeutic regimen or protocol. An amount sufficient or an amount effective refers to sufficiency or effectiveness in a given subject, not a group or the general population. Such amounts will depend in part upon the disease or disorder treated, such as the type or stage (early or advanced) of the disorder or disease, the therapeutic effect desired, as well as the individual subject (e.g., the bioavailability within the subject, gender, age, etc.).

An amount sufficient for the methods, uses and compositions of the embodiments include a total amount of active (e.g., L-tyrosine compound and/or L-serine compound) of about 1-5 grams (g), 5-10 g, 10-15 g, 15-20 g, 20-25 g, 25-30 g, 30-40 g, 40-50 g, 50-75 g or 75-100 g, daily. Amounts sufficient can also be used according to the mass of a subject (e.g., in Kilograms, kg). For example, for a human subject, amounts of L-tyrosine, or a precursor, derivative or conjugate of L-tyrosine, and/or L-serine, or a salt, metabolic precursor, derivative or conjugate of L-serine include about 0.5-10 mg/kg body weight, 10-25 mg/kg body weight, 25-50 mg/kg body weight, 50-100 mg/kg body weight, 100-250 mg/kg body weight, 250-500 mg/kg body weight, 500-750 mg/kg body weight, 750-1,000 mg/kg body weight, 1-5 g/kg body weight, or 5-10 g/kg body weight of a subject. Such amounts for the methods, uses and compositions of the invention can be less, for example, from about 50-500, 500-5000, 5000-25,000 or 25,000-50,000 mg/kg.

Methods and uses of the invention may be practiced prior to (i.e. prophylaxis) or after symptoms begin, before or after symptoms or the disease or disorder develop. Administering L-tyrosine, or a precursor, derivative or conjugate of L-tyrosine, and/or L-serine, or a salt, metabolic precursor, derivative or conjugate of L-serine prior to or immediately following development of a symptom may decrease the severity or frequency of symptoms, or the underlying cause of the neurological disorder, in the subject. In addition, administering L-serine, or a precursor, derivative or conjugate of L-tyrosine, and/or L-serine, or a salt, metabolic precursor, derivative or conjugate of L-serine prior to or immediately following development of one or more symptoms may stabilize or slow progression or worsening of a symptom, or the underlying cause of the neurological disorder or disease.

Methods and compositions of the invention may be used in vitro, ex vivo or in vivo. Compositions can be administered or delivered as a single or multiple dosage form, on consecutive or alternating days or intermittently, to a subject. For example, single or multiple dosage forms can be administered or delivered on alternating days or intermittently, over about 1 to 7, or 7 to 45, or 45 to 90 days or over about 1-4, 4-8, 8-12, 12-18, 18-24, 24-48, or more weeks, to a subject. Compositions also can be administered on a daily basis, multiple times per day to achieve a total daily dosage for a period of time, then administered more frequently on a daily basis, multiple times per day to achieve a greater total daily dosage, and this process can be repeated one or more times until a final desired daily dosage amount is achieved by the administration.

The term “contacting” means direct or indirect binding or interaction between two or more entities (e.g., between L-tyrosine, or a precursor, derivative or conjugate of L-tyrosine, and/or L-serine, a precursor, derivative or conjugate of L-serine, or molecule target, within a cell, for example). Contacting as used herein includes in solution, in solid phase, in vitro, ex vivo, in a cell and in vivo. Contacting in vivo can be referred to as administering, or administration, or in vivo delivery.

The term “subject” refers to animals, typically mammalian animals, such as a non-human primate (vervets, gorillas, chimpanzees, orangutans, macaques, gibbons), a domestic animal (dogs and cats), a farm animal (horses, cows, goats, sheep, pigs), experimental animal (mouse, rat, rabbit, guinea pig) and humans. Human subjects include adults and children. Human subjects include those having or at risk of having a neurological disorder. At risk subjects can be identified through genetic screening for predisposition towards a neurological disorder or a family history of a neurological disorder, for example, by accumulation of BMAA in keratinaceous tissues or blood plasma (see, e.g., U.S. Pat. Nos. 7,256,002 and 7,670,783). Subjects further include disease model animals (e.g., such as mice and non-human primates) for testing in vivo efficacy of L-serine, or a precursor, derivative or conjugate of L-serine.

The embodiments also provide compositions, including L-tyrosine, or a precursor, derivative or conjugate of L-tyrosine, and/or L-serine, or a salt, metabolic precursor, derivative or conjugate of L-serine in amounts that are able to produce one or more of the activities associated with L-tyrosine and/or L-serine. In one embodiment, a composition includes L-tyrosine, or a precursor, derivative or conjugate of L-tyrosine, and/or L-serine, or a salt, metabolic precursor, derivative or conjugate of L-serine in amounts sufficient to treat a neurological disorder. In another embodiment, a composition includes an amount of L-tyrosine, or a precursor, derivative or conjugate of L-tyrosine, and/or an amount of L-serine, or a salt, metabolic precursor, derivative or conjugate of L-serine sufficient to inhibit, reverse, ameliorate or reduce one or more symptoms of a neurological disorder. In yet another embodiment, a composition includes an amount of L-tyrosine, or a precursor, derivative or conjugate of L-tyrosine, and/or an amount of L-serine, or a salt, metabolic precursor, derivative or conjugate of L-serine sufficient to reverse an underlying causes of a neurological disorder.

Amounts of L-tyrosine, or a precursor, derivative or conjugate of L-tyrosine, and/or amounts of L-serine, or a salt, metabolic precursor, derivative or conjugate of L-serine for the methods, uses and compositions of the invention include about 1-10 milligrams (mg), 10-25 mg, 25-50 mg, 50-100 mg, 100-250 mg, 250-500 mg, 500-750 mg, 750-1,000 mg, 1,000-2,000 mg, 2,000-3,000 mg, 3,000-4,000 mg, 4,000-5,000 mg, 5,000-7,500 mg, 7,500-10,000 mg, 10-15 grams (g), 15-20 g, 20-25 g, 25-30 g, 30-40 g, 40-50 g, 50-75 g or 75-100 g of each individual component. Amounts can also be produced and/or provided according to the mass of a subject (e.g., in Kilograms, kg). For example, for a human subject, amounts of L-tyrosine, or a precursor, derivative or conjugate of L-tyrosine and/or amounts of L-serine, or a salt, metabolic precursor, derivative or conjugate of L-serine can be adjusted according to the mass of the human. Such amounts of L-tyrosine, or a precursor, derivative or conjugate of L-tyrosine, and/or L-serine, or a salt, metabolic precursor, derivative or conjugate of L-serine include about 1-10 mg/kg body weight, 10-25 mg/kg body weight, 25-50 mg/kg body weight, 50-100 mg/kg body weight, 100-250 mg/kg body weight, 250-500 mg/kg body weight, 500-750 mg/kg body weight, 750-1,000 mg/kg body weight, 1-5 g/kg body weight, or 5-10 g/kg body weight of a subject. Such amounts can be less, for example, from about 50-500, 500-5000, 5000-25,000 or 25,000-50,000 ng/kg.

The amounts of each respective component, the L-tyrosine component and the L-serine component, if combined, can vary with the amount of the L-tyrosine component typically being greater than the amount of the L-serine component. For example, in an embodiment, the composition is in the form of a tablet, capsule, gel capsule, gummy, or jelly bean, and the amount of the L-tyrosine component is administered in a dosage of from about 15 to about 35 grams/day, or from about 20 to about 30 grams/day, or about 25 grams/day, and the amount of the L-serine component is administered in a dosage of from about 1 to about 10 grams/day, or from about 3 to about 7 grams/day, or about 5 grams/day. The total amount of the combined dosages can be about 30 grams/day. In an embodiment, the composition is in the form of a tablet, capsule, gel capsule, gummy, or jelly bean, and the amount of the L-tyrosine component in the composition is from about 0.4 to about 1.2 grams, or from about 0.5 to about 1 grams, or about 0.83 grams, and the amount of the L-serine component in the composition is from about 0.1 to about 0.3 grams, or from about 0.15 to 0.24 grams, or about 0.17 grams.

To achieve a desired total daily dosage, an embodiment includes administration of from 5-50 or from about 10 to 40 or about 30 forms of the composition (e.g., tablet, capsule, gel capsule, gummy, or jelly bean) per day. In a preferred embodiment, the composition is in the form of a jelly bean comprising about 0.83 grams of the L-tyrosine component and about 0.17 grams of the L-serine component, if used, (each jelly bean containing about 1 gram of active substance) such that the total daily dosage, if the subject is administered 30 jelly beans per day, is about 25 grams of the L-tyrosine component and about 5 grams of the L-serine component. In one embodiment, a subject is administered the above composition from about 5 to about 15 or about 10 times per day for 1-4 weeks, or 2 weeks, and then the subject is administered the above composition from about 15 to about 30 or about 20 times per day for 1-4 weeks, or 2 weeks, and then the subject is administered the above composition about 30 times per day thereafter.

Methods and uses of the invention can be practiced using L-tyrosine, or a precursor, derivative or conjugate of L-tyrosine, and/or L-serine, or a salt, metabolic precursor, derivative or conjugate of L-serine, optionally in such amounts as set forth herein. A derivative or conjugate of L-tyrosine can be a modification (e.g., a protecting group) at any one, any two or all three functional groups, namely, the amino moiety (NH₂), acid moiety (—COOH) and/or the hydroxyl moiety (—OH) groups) of L-tyrosine. The modified groups can be cleaved in vivo to yield free L-tyrosine and a hydrolyzed protecting group. Suitable groups are pharmaceutically acceptable and typically substantially non-toxic. A derivative or conjugate of L-serine may have enhanced stability and/or solubility compared to free L-tyrosine which aids in storage and dosing, and uptake of the L-tyrosine active compound. These and other L-tyrosine derivatives and conjugates known to one of skill in the art are included in the invention methods, uses and compositions (e.g., pharmaceutical formulations).

Non-limiting examples of L-tyrosine derivatives and conjugates in which a hydroxyl moiety is derivatized to form a protected hydroxyl include ester, carbonate, phosphate, and sulfonate ester. The hydroxyl group of L-tyrosine can be selectively esterified with a suitable carbonyl, phosphonyl, or sulfonyl electrophile after prior protection of the amino and acid moieties of the L-tyrosine. Typically, one or both of the amino and acid protecting groups can be removed after selective esterification of the hydroxyl moiety; bio-compatibility of the acid and amino protecting groups is not an issue where both protecting groups are removed. Non-limiting examples of amino protecting groups include but are not limited to tert-butoxycarbonyl (Boc) and carbobenzyloxy (CBz), which are removable under acidic and hydrogenolysis conditions, respectively. Suitable acid protecting groups include tert-butyl (tBu) and benzyl (Bn) esters, which are removable under acidic and hydrogenolysis conditions, respectively. Suitable hydroxyl protection groups, are esters of carboxylic acids, phosphoric acid, phosphonic acids, sulfuric and sulfonic acids and other hydroxyl protecting groups known to the skilled artisan. One of ordinary skill in the art will appreciate that hydroxyl-protected L-tyrosine may exist as a zwitterionic species as in the free amino acid or may be readily converted to a protic acid addition salt.

Non-limiting examples of L-tyrosine derivatives and conjugates in which the amino moiety is derivatized include formation of an amide or urea, or carbamate-containing L-tyrosine. The amino moiety of L-tyrosine can be selectively derivatized after prior protection of the hydroxyl and acid moieties of the L-tyrosine. Both hydroxyl and acid moieties in L-tyrosine can be protected with groups amenable to removal after the final derivatization of the amino group. Typically, one or both of the hydroxyl and acid moiety protecting groups are removed after selective derivatization of the L-tyrosine amino moiety.

L-tyrosine derivatives and conjugates include derivatization of the acid moiety, which can be selectively derivatized after protecting the hydroxyl and amino moieties of the L-tyrosine. Both hydroxyl and amino moieties in L-tyrosine can be masked with protecting groups that are amenable to removal after the final protection of the acid moiety. Typically, one or both of the hydroxyl and amino moiety protecting groups are removed after selective derivatization of the L-tyrosine acid moiety.

In various additional embodiments, an L-tyrosine conjugate includes a polymer. For example, L-tyrosine can be within a small (e.g., 2-10) residue peptide which includes other amino acids. In this embodiment, enzymes such as peptidases and pepsin can cleave the peptide into individual amino acids, releasing L-tyrosine. In such embodiments, the peptide can include two, three, four, five, six, seven, eight, nine, ten, 11, 12, 13, 14, 15, 16, 17, 18, 19, 20, etc., or more L-tyrosine molecules Tyr-[Tyr]n where n is 1 to 1000. The administered peptide yields serine or smaller serine peptides which are in turned hydrolyzed.

Starting materials useful for preparing L-tyrosine derivatives and conjugates are commercially available or can be prepared by well-known synthetic methods (Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al, “Reagents for Organic Synthesis,” Volumes 1-17, Wiley Interscience; Trost et al, “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995). Other methods for synthesis of hydroxyl-protected tyrosine compound and amino acid protection groups will be readily apparent to the skilled artisan.

L-tyrosine, and precursors, derivatives and conjugates of L-tyrosine, and/or L-serine, or a salt, metabolic precursor, derivative or conjugate of L-serine and compositions thereof (e.g., pharmaceutical formulations), may be administered systemically, regionally or locally by any route. For example, L-tyrosine, or a precursor, derivative or conjugate of L-tyrosine, and/or L-serine, or a salt, metabolic precursor, derivative or conjugate of L-serine may be administered intravenously, orally (e.g., ingestion), intracranially, intraspinally, intramuscularly, intraperitoneally, intradermally, subcutaneously, intracavity, transdermally (topical), parenterally, e.g. transmucosal and rectally. Methods, uses of L-tyrosine, and precursors, derivatives and conjugates of L-tyrosine, and/or L-serine, or a salt, metabolic precursor, derivative or conjugate of L-serine and compositions thereof, of the embodiments including pharmaceutical formulations can be administered via a microencapsulated delivery system or packaged into an implant for sustained, continuous or intermittent administration.

Compositions further include pharmaceutical formulations containing L-tyrosine, or a precursor, derivative or conjugate of L-tyrosine and/or L-serine, or a salt, metabolic precursor, derivative or conjugate of L-serine. Such pharmaceutical formulations can be formulated in an amount having one or more of the activities disclosed herein, and a pharmaceutically acceptable carrier or excipient. In various embodiments, a pharmaceutical formulation includes L-tyrosine, or a precursor, derivative or conjugate of L-tyrosine and/or L-serine, or a salt, metabolic precursor, derivative or conjugate of L-serine in amounts sufficient to achieve an intended effect.

As used herein, the terms “pharmaceutically acceptable” and “physiologically acceptable” refer to carriers, excipients, diluents and the like that can be administered to a subject, preferably without producing excessive adverse side-effects (e.g., nausea, abdominal pain, headaches, etc.). Such preparations for administration include sterile aqueous or non-aqueous solutions, suspensions, and emulsions.

Pharmaceutical formulations can be made from carriers, diluents, excipients, solvents, dispersion media, coatings, antibacterial and antifungal agents, isotonic and absorption delaying agents, and the like, compatible with administration to a subject. Such formulations can be contained in a tablet (coated or uncoated), capsule (hard or soft), microbead, emulsion, powder, granule, crystal, suspension, syrup or elixir. Supplementary active compounds and preservatives, among other additives, may also be present, for example, antimicrobials, anti-oxidants, chelating agents, and inert gases and the like.

A pharmaceutical formulation can be formulated to be compatible with its intended route of administration. Thus, pharmaceutical formulations include carriers, diluents, or excipients suitable for administration by routes including intraperitoneal, intradermal, subcutaneous, oral (e.g., ingestion or inhalation), intravenous, intracavity, intracranial, intraspinal, transdermal (topical), parenteral, e.g. transmucosal and rectal.

Solutions or suspensions used for parenteral, intradermal, or subcutaneous application can include the following: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical formulations suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. Fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Isotonic agents, for example, sugars, polyalcohols such as manitol, sorbitol, sodium chloride can be included in the composition. Prolonged absorption of injectable formulations can be achieved by including an agent that delays absorption, for example, aluminum monostearate or gelatin.

For oral administration, L-tyrosine, or a precursor, derivative or conjugate of L-tyrosine, and/or L-serine, or a salt, metabolic precursor, derivative or conjugate of L-serine or compositions thereof, can be incorporated with excipients in the form of tablets, troches, or capsules, e.g., gelatin capsules, gummies, and jelly beans. Pharmaceutically compatible binding agents, and/or adjuvant materials can be included in oral formulations. The tablets, pills, capsules, troches, gummies, jelly beans, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidani such as colloidal silicon dioxide; a sweetening agent such as sucrose or saccharin; or a flavoring agent such as peppermint, methyl salicylate, or flavoring.

Formulations can also include carriers to protect L-tyrosine, or a precursor, derivative or conjugate of L-tyrosine and/or L-serine, or a salt, metabolic precursor, derivative or conjugate of L-serine, against degradation or elimination from the body, such as a controlled release formulation, including materials that slowly degrade within the body and in turn release the active ingredient(s). For example, a time delay material such as glyceryl monostearate or glyceryl stearate alone, or in combination with a wax, may be employed.

Additional formulations include biodegradable or biocompatible particles or polymeric substances such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. Methods for preparation of such formulations are known to those skilled in the art. The materials can also be obtained commercially from Alza Corporation and Nova Pharmaceuticals, Inc., for example.

The rate of release of L-tyrosine, or a precursor, derivative or conjugate of L-tyrosine and/or L-serine, or a salt, metabolic precursor, derivative or conjugate of L-serine, can be controlled by altering the concentration or composition of such macromolecules. For example, L-tyrosine, or a precursor, derivative or conjugate of L-tyrosine and/or L-serine, or a salt, metabolic precursor, derivative or conjugate of L-serine, can be entrapped in microcapsules prepared by coacervation techniques or by interfacial polymerization, for example, by the use of hydroxymethylcellulose or gelatin-microcapsules or poly(methylmethacrolate) microcapsules, respectively, or in a colloid drug delivery system. Colloidal dispersion systems include macromolecule complexes, nano-capsules, microspheres, microbeads, and lipid-based systems including oil-in-water emulsions, micelles, mixed micelles, and liposomes. These can be prepared according to methods known the skilled artisan, for example, as described in U.S. Pat. No. 4,522,811.

Additional pharmaceutical formulations appropriate for administration are known in the art and are applicable in the methods, uses and compositions of the invention (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; and Pharmaceutical Principles of Solid Dosage Forms, Technonic Publishing Co., Inc., Lancaster, Pa., (1993)).

L-tyrosine, or a precursor, derivative or conjugate of L-tyrosine and/or L-serine, or a salt, metabolic precursor, derivative or conjugate of L-serine, of the embodiments can include combinations of other compositions, and be included in the pharmaceutical compositions of the invention. For example, a drug that is used to treat a neurological disorder can be included with L-tyrosine, or a precursor, derivative or conjugate of L-tyrosine and/or L-serine, or a salt, metabolic precursor, derivative or conjugate of L-serine.

L-tyrosine, or a precursor, derivative or conjugate of L-tyrosine, and/or L-serine, or a salt, metabolic precursor, derivative or conjugate of L-serine, including pharmaceutical formulations thereof can be packaged into kits, which optionally can contain instructions for use, for example, practicing a method or use of the invention. The invention therefore provides kits. In one embodiment, a kit includes L-tyrosine, or a precursor, derivative or conjugate of L-tyrosine, and/or L-serine, or a salt, metabolic precursor, derivative or conjugate of L-serine, and/or a pharmaceutical formulation, packaged into suitable packaging material. In additional embodiments, a kit includes a label or packaging insert for practicing a method of the invention. Thus, in one embodiment, a kit includes instructions for treating a subject having or at risk of having a neurological disorder, in vitro, in vivo, or ex vivo. In additional embodiments, a kit includes a label or packaging insert including instructions for treating a subject having a neurological disorder in vivo, or ex vivo.

As used herein, the term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method or use of the invention. Kits of the invention therefore can additionally include instructions for using the kit components in a method or use of the invention.

Instructions can include instructions for practicing any of the methods or uses of the invention described herein. Thus, L-tyrosine, or a precursor, derivative or conjugate of L-tyrosine and/or L-serine, or a salt, metabolic precursor, derivative or conjugate of L-serine, and pharmaceutical compositions thereof can be included in a container, pack, or dispenser together with instructions for administration to a subject. Instructions may additionally include indications, a satisfactory clinical endpoint, any adverse symptoms that may occur, or additional information required by the Food and Drug Administration for use on a human subject.

The instructions may be on “printed matter,” e.g., on paper or cardboard within the kit, on a label affixed to the kit or packaging material, or attached to a vial or tube containing a component of the kit. Instructions may comprise voice or video tape which can optionally be included on a computer readable medium, such as a disk (hard disk), optical CD such as CD- or DVD-ROM/RAM, magnetic tape, electrical storage media such as RAM and ROM and hybrids of these such as magnetic/optical storage media.

Kits can also include one or more drugs that provide a synergistic or additive effect or that reduce or ameliorate one or more symptoms of a neurological disorder. For example, a drug that reduces or decreases a symptom of a neurological disorder may be included. Invention kits can additionally include a buffering agent, a preservative, or a stabilizing agent. The kit can further include control components for assaying an activity or effect of treatment. Each component of the kit can be enclosed within a separate individual container. For example, a kit can include a single unit dose of L-tyrosine, or a precursor, derivative or conjugate of L-tyrosine and/or L-serine, or a salt, metabolic precursor, derivative or conjugate of L-serine, as set forth herein. Alternatively, a kit can include multiple unit doses of L-tyrosine, or a precursor, derivative or conjugate of L-tyrosine and/or L-serine, or a salt, metabolic precursor, derivative or conjugate of L-serine. For example, each of the multiple unit doses would contain an amount of L-tyrosine, or precursor, derivative or conjugate of L-tyrosine, and/or L-serine, or a salt, metabolic precursor, derivative or conjugate of L-serine, in a separate individual container. Kit components can be in a mixture of one or more containers and all of the various containers can be within single or multiple packages.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All patents, applications, publications, other references, GenBank citations and ATCC citations cited herein are expressly incorporated by reference herein in their entirety. In case of conflict, the specification, including definitions, will control.

As used herein, the singular forms “a”, “and,” and “the” include plural referents unless the context clearly indicates otherwise. Thus, for example, reference to “L-serine, or a precursor, derivative or conjugate of L-serine” includes a plurality of L-serine, or precursors, derivatives or conjugates of L-serine, and so forth.

As used herein, all numerical values or numerical ranges include integers within such ranges and fractions of the values or the integers within ranges unless the context clearly indicates otherwise. Thus, for example, reference to a range of 1-10, includes 1, 2, 3, 4, 5, etc., as well as 1.1, 1.2, 1.3, 1.4, 1.5, etc., 2.1, 2.2, 2.3, 2.4, 2.5, etc., and so forth. A reference to a range includes a reference to subranges within that range. Thus, for example, reference to 1-10 also include 1-3, 1-4, 1-5, 1-6, 2-4, 2-5, 2-6, 2-7, 3-5, 3-6, 3-7, 3-8, etc. Reference to a series of ranges, for example, reference to a range of 1-10 mg, 10-25 mg, 25-50 mg, 50-100 mg, 100-250 mg, 250-500 mg, 500-750 mg, 750-1,000 mg, 1,000-2,000 mg, 2,000-3,000 mg, 3,000-4,000 mg, 4,000-5,000 mg, 5,000-7,500 mg, 7,500-10,000 mg, 10-15 g, 15-20 g, 20-25 g, 25-30 g, 30-40 g, 40-50 g, 50-75 g and 75-100 g include combinations of combined ranges, such as 10-50, 50-500, 70-100 mg or g, etc. A series of ranges include both lower and upper ends of those ranges combined into ranges. Thus, for example, reference to a series of ranges such as 50-100 100-200, and 200-300, includes a range of 50-200, 50-300, 100-300, etc.

The embodiments are generally disclosed herein using affirmative language to describe the numerous embodiments. The embodiments also specifically include features in which particular subject matter is excluded, in full or in part, such as substances or materials, method steps and conditions, protocols, procedures, assays or analysis. A number of embodiments of the invention have been described.

Nevertheless, it will be understood that various modifications may be made without departing from the spirit and scope hereof. Accordingly, the following examples are intended to illustrate but not limit the scope of embodiments.

### EXAMPLES

### Experiments 1 and 2

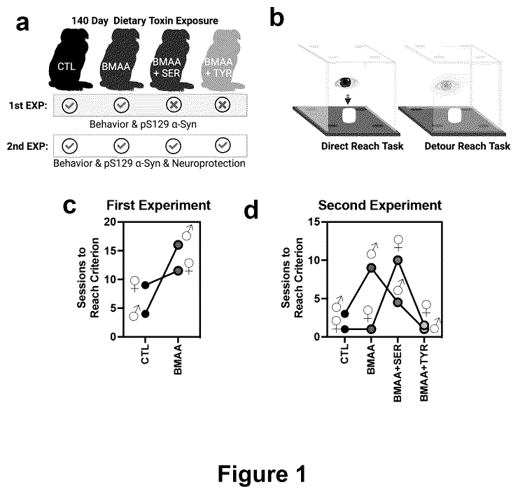

The cognitive ability of marmosets in four different cohorts of the second experiment using the Detour-Reaching Task (DRT) were studied after observing BMAA's effect on cohorts in the first experiment (FIG. 1 a-c). Animals were assessed as to the number of sessions it took to reach criterion, which was defined as 90% successful food reward retrieval in less than 6 secs on average, which indicates strong inhibitory control (Error! Bookmark not defined.). Because of the right-skewness of the session counts, our analysis of variance used the logarithm of sessions required as the response variable. Cohorts had significantly different mean numbers of sessions needed to reach criterion (p=0.011), with the BMAA plus L-tyrosine group performing best, followed by the control, BMAA, and BMAA plus L-serine groups, respectively. The interaction between group and sex was also significant (p=0.021), indicating that the relative effects of the group assignment was different between males and females (FIG. 1d). Males and females co-administered with L-tyrosine were able to meet criteria for the DTR with a mean minimum number of sessions required, 1.0 and 1.5 sessions required, respectively.

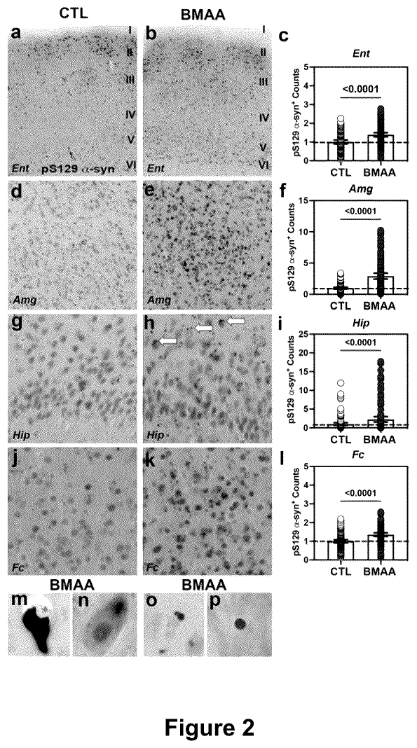

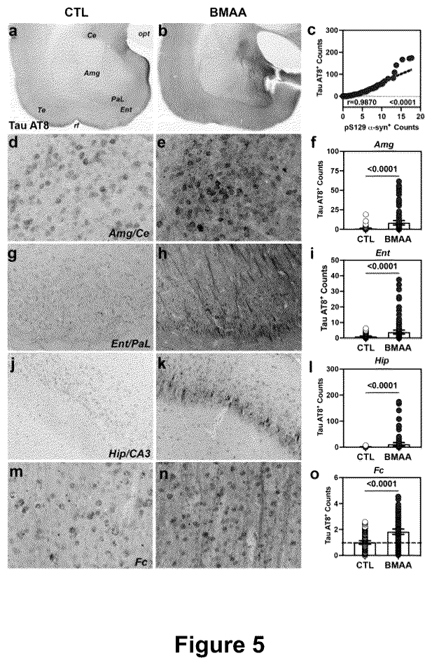

In a first experiment, it was discovered that chronic dosing with BMAA induced region-specific increases in pS129 α-syn expression, with its effects on the amygdala and hippocampus being the most pronounced (p<0.0001) (FIG. 2). Phosphorylated S129 α-syn observed in the BMAA cohorts consisted of diffused and focal intracellular α-syn staining, aggregates, occurring with rare to sparse LBs and focal Lewy neurites (FIG. 2). M. Goedert, et al., 100 years of Lewy pathology. Nature reviews. Neurology 9, 13-24 (2013); M. Sakamoto et al., Heterogeneity of nigral and cortical Lewy bodies differentiated by amplified triple-labeling for alpha-synuclein, ubiquitin, and thiazin red. Exp. Neurol. 177, 88-94 (2002). BMAA exposure has been shown to trigger tauopathies in non-human primates (Cox, et al., infra). Here BMAA also increased AT8, a marker of paired helical filaments (PHF) tau expression, in the same brain regions from 80% to 1,050% (p<0.0001) (FIG. 5). The ranked expression of pS129 α-syn in toxin exposed animals was positively correlated with tau-AT8 expression (p<0.0001), suggesting a 1:1 ratio in their expression.

In the second experiment, it was discovered that BMAA had region specific effects and that the means for total pS129 α-syn expression across all brain regions significantly differed between the treatment cohorts (p=0.004, as shown in Table 1 below.

TABLE 1
Phosphorylated Serine 129 α-synuclein (means are given for each treatment group × sex combination)

| | | | Males | | | | Females | | | |
|---|---|---|---|---|---|---|---|---|---|---|
| | Brain | | | | BMAA + | BMAA + | | | BMAA + | BMAA + |
| No | Region | ROI | CTL | BMAA | SER | TYR | CTL | BMAA | SER | TYR |
| 1 | Frontal | A8 | 1.5 | 7.6 | 3.3 | 0.1 | 2.6 | 0.9 | 2.2 | 0 |
| 2 | Amygdala | BLD | 3.5 | 3.2 | 0.7 | 0.1 | 6.4 | 0.8 | 0 | 0 |
| 3 | MidBrain | SN | 4.1 | 1 | 0.4 | 0 | 0.2 | 0.2 | 0.7 | 0.5 |
| 4 | Occipital | V1 | 4.5 | 0.1 | 0 | 0 | 0.1 | 0 | 0 | 0 |
| 5 | Frontal | A11 | 8.1 | 7.9 | 17.9 | 6.4 | 6.2 | 6 | 3.1 | 0 |
| 6 | Parietal | PE | 8.7 | 15.9 | 11.8 | 0.7 | 5.2 | 3.8 | 2.3 | 0 |
| 7 | Amygdala | PAL | 9.1 | 7.6 | 5.4 | 1.6 | 11.4 | 8.7 | 0.4 | 0.1 |
| 8 | Spinal cord | Post H | 9.1 | 11.9 | 19.1 | 4.2 | 10.8 | 26.9 | 7.5 | 1.9 |
| 9 | Spinal cord | Ant H | 17.2 | 29.4 | 51.2 | 8.6 | 19.3 | 19.8 | 16.4 | 0.5 |
| 10 | Occipital | V2 | 18.6 | 15.6 | 13.8 | 1.9 | 7.8 | 2.3 | 0.5 | 0 |
| 11 | Temporal | AuCrtx | 20.2 | 11.3 | 16 | 9.2 | 5.8 | 9.9 | 1.9 | 0 |
| 12 | Entorhinal | pENT | 20.9 | 17 | 15.2 | 2.6 | 15.6 | 19.6 | 9.8 | 0.1 |
| 13 | Hippocampus | CA3 | 23.8 | 44.8 | 22.8 | 4 | 14.8 | 0 | 2.6 | 4.4 |
| 14 | Brainstem | Med4V | 34.8 | 43.9 | 36.9 | 9.3 | 24.7 | 14.9 | 23.5 | 0 |
| 15 | Entorhinal | aENT | 49.9 | 48.9 | 44.4 | 17.4 | 18.1 | 14.8 | 29.3 | 0.1 |
| 16 | Brainstem | (MLF) | 58.5 | 42 | 49.1 | 9.9 | 34.3 | 34.1 | 7.5 | 0 |
| | | Mean | 18.3 | 19.3 | 19.3 | 4.8 | 11.5 | 10.2 | 6.7 | 0.5 |
| | | Lower | 9.4 | 10.3 | 10.0 | 2.1 | 6.5 | 4.5 | 2.0 | −0.1 |
| | | 95% CI | | | | | | | | |
| | | Upper | 27.2 | 28.2 | 28.5 | 7.4 | 16.4 | 15.8 | 11.5 | 1.1 |
| | | 95% CI | | | | | | | | |
| | | (All ROI) | CTL | BMAA | BMAA + | BMAA + | Male | Female | | |
| | | M + F | | | SER | TYR | | | | |
| | | Mean | 14.9 | 14.7 | 13.0 | 2.6 | 15.4 | 7.2 | | |
| | | Lower | 9.9 | 9.5 | 7.6 | 1.1 | 11.4 | 4.9 | | |
| | | 95% CI | | | | | | | | |
| | | Upper | 19.8 | 20.0 | 18.4 | 4.1 | 19.3 | 9.5 | | |
| | | 95% CI | | | | | | | | |

The means for the Control, BMAA, and BMAA plus L-serine groups across all brain regions were 14.9, 14.7, and 13.0, respectively, while the mean of the BMAA plus L-tyrosine cohort was much lower at 2.6, with significant Tukey pairwise comparisons with the other three groups (p=0.006, 0.007, and 0.016, respectively, as shown in Table 2 below:

TABLE 2
Tukey Pairwise Mean α-synuclein Comparison

| | diff | lwr | upr | p adj |
|---|---|---|---|---|
| BMAA-Control | −0.1519097 | −8.404611 | 8.100792 | 0.9999199 |
| LserineBMAA- | −1.8758681 | −10.128569 | 6.376833 | 0.8832938 |
| Control | | | | |
| LtyrosineBMAA- | −12.2500000 | −20.502701 | −3.997299 | 0.0062699 |
| Control | | | | |
| LserineBMAA- | −1.7239583 | −9.976660 | 6.528743 | 0.9058841 |
| BMAA | | | | |
| LtyrosineBMAA- | −12.0980903 | −20.350792 | −3.845389 | 0.0067509 |
| BMAA | | | | |
| LtyrosineBMAA- | −10.3741319 | −18.626833 | −2.121431 | 0.0160933 |
| LserineBMAA | | | | |

In general, male animals had over twice the density of pS129 α-syn (15.4) compared to females (7.2) (p=0.002, values are the average of the 4 means for males and females, respectively, from Table 1). When examining regions of interests (ROIs) involved in the Detour Reach Task (DRT), pS129 α-syn expression increased in parallel with cognitive test scores, especially in the frontal cortex (Fc) which had twice the expression of pS129 α-syn. L-tyrosine co-administration reduced the pS129 α-syn to near that of the control cohort, as shown in Table 3 below:

TABLE 3
Detour Reach Task test scores and pS129 α-synuclein
tissue expression from the second experiment

| | | | BMAA + | BMAA + |
|---|---|---|---|---|
| | CTL | BMAA | SER | TYR |
| DTR Scores | 1.0 | 2.5 | 3.6 | 0.6 |
| | (−0.6-2.6) | (−1.2-6.2) | (−0.6-7.9) | (0.2-1.0) |
| Brain ROIs | | | | |
| All 16 | 1.0 | 1.0 | 0.9 | 0.9 |
| ROIs | (0.5-1.5) | (0.6-1.4) | (0.4-1.3) | (0.4-1.3) |
| Frontal | 1.0 | 2.1 | 1.3 | 1.3 |
| cortex | (−0.2-2.2) | (−1.9-6.1) | (−0.2-2.9) | (−0.2-2.9) |
| Hippocampus | 1.0 | 1.2 | 0.7 | 0.7 |
| | (−0.9-2.9) | (−1.0-3.3) | (−0.8-2.1) | (−0.8-2.1) |
| Parietal | 1.0 | 1.4 | 1.0 | 1.0 |
| cortex | (0.4-1.6) | (−0.2-3.1) | (−0.3-2.4) | (−0.3-2.4) |
| Spinal | 1.0 | 2.0 | 1.3 | 1.3 |
| cord | (0.0-2.0) | (−0.1-4.0) | (−0.1-2.8) | (−0.1-2.8) |

Normalized scores and counts to control mean and (95% confidence interval) are given for each treatment group; (N = 4 marmoset/group)

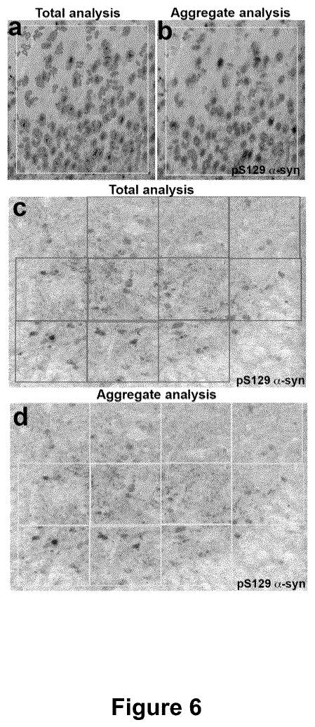

To further evaluate BMAA's effects on pS129 α-syn expression, a specialized threshold to capture only extracellular aggregates and exclude those that are located in the soma of neurons was applied to brain regions associated with DRT execution and PD (FIG. 6). The analysis showed that chronic dietary exposure to BMAA increased the average α-syn aggregates as much as 380% in some brain regions (see Table 4 below).

TABLE 4
α-synuclein aggregate analysis from second experiment

| | | | BMAA + | BMAA + |
|---|---|---|---|---|
| Brain ROIs | CTL | BMAA | SER | TYR |
| Frontal cortex | 1.0 | 3.2 | 1.3 | 0.1 |
| Substantia nigra | 1.0 | 4.8 | 2.5 | 0.3 |
| Hippocampus | 1.0 | 1.6 | 0.8 | 0.3 |
| Spinal cord | 1.0 | 1.6 | 0.8 | 0.3 |
| Visual cortex | 1.0 | 0.1 | 1.2 | 0.0 |
| Mean | 1.0 | 2.3 | 1.3 | 0.2 |
| (95% CI): | (0.6-1.4) | (0.5-4.0) | (0.6-2.0) | (0.1-0.3) |

Normalized counts to the control mean for 10 microscopic scan fields for each anatomical ROI are given for each treatment group; (N = 4 marmoset/ group)

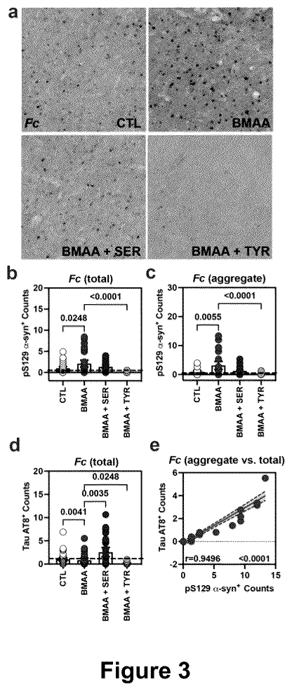

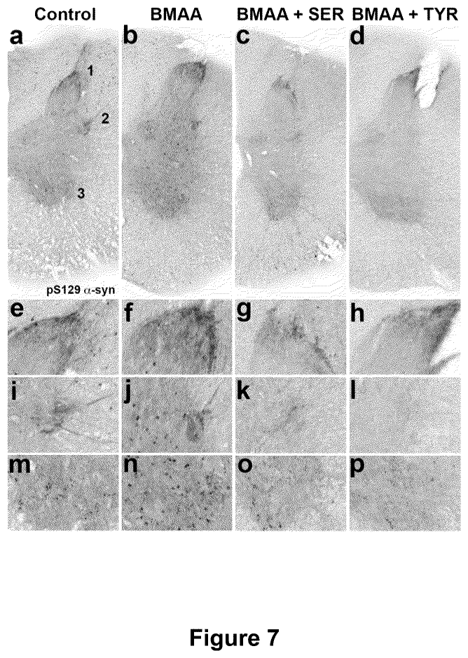

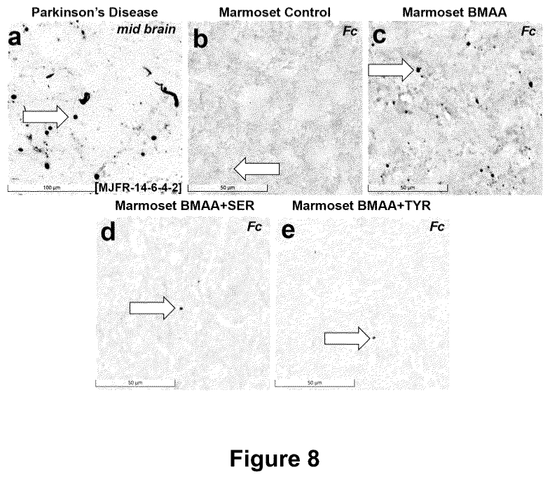

In the spinal cord, a target region of BMAA, pS129 α-syn aggregate expression was increased in both the anterior, lateral and posterior horns and displayed morphology and anatomical distribution reminiscent to those described by Braak in autopsy specimens from PD patients (FIG. 7). K. Del Tredici, H. Braak, Spinal cord lesions in sporadic Parkinson's disease. Acta neuropathologica 124, 643-664 (2012). Co-administration of L-tyrosine with BMAA decreased α-syn aggregate formation, including the frontal cortex (−80%), which supports the observed improved cognitive performance in the DRT (FIG. 1C, FIG. 3a, c, Table 4). L-tyrosine's effects on the Fc were confirmed using a confirmation specific anti-α-syn aggregate antibody (FIG. 8).

Lastly, L-tyrosine also decreased the amount of PHF tau deposits in the Fc (FIG. 3d). However, those in the BMAA plus L-tyrosine group had greater than a 90% reduction in PHF tau compared to the BMAA group (p=0.0049). We found that PHF tau deposits were most abundant in males (p=0.0013), and that group by sex interaction was also significant (p=0.0087), with males exhibiting comparably low PHF tau measures in the L-tyrosine group when compared to females, but much larger PHF tau measures than females in the other groups. For both sexes, density of PHF tau aggregates were lowest in the BMAA plus L-tyrosine group (mean=0.31), as shown in Table 5 below:

TABLE 5
Paired helical filament tau
AT8⁺ Aggregates from Second Experiment

| | | | BMAA + | BMAA + |
|---|---|---|---|---|
| Brain ROIs | CTL | BMAA | SER | TYR |
| Frontal cortex | 1.0 | 0.8 | 2.6 | 0.1 |
| Hippocampus | 1.0 | 0.9 | 1.7 | 0.1 |
| Entorhinal cortex | 1.0 | 0.8 | 2.3 | 0.1 |
| Amygdala | 1.0 | 0.2 | 0.7 | 0.1 |
| Mean | 1.0 | 0.7 | 1.8 | 0.1 |
| (95% CI) | (0.4-1.6) | (0.1-1.2) | (0.6-3.0) | (0.0-0.1) |

Normalized counts to the control mean for 10 microscopic scan fields for each anatomical ROI are given for each treatment group; (N = 4 marmoset/group)

At the presynapse, α-syn regulates the release of dopamine, a neurotransmitter that is critical for motor function. In PD, α-syn modulation of dopamine release is impaired due to protein misfolding and aggregation followed by a loss of dopaminergic neurons. P. Calabresi et al., Alpha-synuclein in Parkinson's disease and other synucleinopathies: from overt neurodegeneration back to early synaptic dysfunction. Cell Death Dis. 14, 176 (2023); R. G. Perez et al., A role for alpha-synuclein in the regulation of dopamine biosynthesis. J. Neurosci. 22, 3090-3099 (2002). In advance stages of PD, a significant decrease in dopamine levels is a prelude to dementia. Here, we found that L-tyrosine protects against the formation of α-syn aggregates, LB and PHF tau, and helps preserve cognitive function in a marmoset model. In our study, male marmosets developed more α-syn aggregates, LBs, and cognitive impairment. However, the reduction in α-syn aggregates mean levels was substantial for both males and females in the L-tyrosine group. Comparing the toxin group to the L-tyrosine group, the mean level of α-syn dropped from 19.2 to 4.7 among males, and from 10.2 to 0.5 among females (Table 1).

This marmoset model of BMAA-induced α-syn misfolding and aggregation and LB formation may show promise for future studies. Marmosets with chronic dietary exposure to BMAA display increased PHF tau deposition (FIG. 5) similar to vervets (Chlorocebus sabaeus), (Cox, et al., infra, and M. Goedert, et al., Monoclonal antibody AT8 recognises tau protein phosphorylated at both serine 202 and threonine 205. Neurosci. Lett. 189, 167-169 (1995). perhaps suggesting that oligomerization of α-syn may be a prelude to tauopathy (FIG. 3d and FIG. 6). S. Moussaud et al., Alpha-synuclein and tau: teammates in neurodegeneration? Mol. Neurodegener. 9, 43 (2014); U. Sengupta et al., Pathological interface between oligomeric alpha-synuclein and tau in synucleinopathies. Biol. Psychiatry 78, 672-683 (2015).

It is noted that PD patients with co-current tauopathies have four times the risk of developing dementia. D. O. Astrom et al., High risk of developing dementia in Parkinson's disease: a Swedish registry-based study. Sci. Rep. 12, 16759 (2022); L. Pan, L. Meng, M. He, Z. Zhang, Tau in the Pathophysiology of Parkinson's Disease. J. Mol. Neurosci. 71, 2179-2191 (2021). PD is twice as common in men than women (S. Cerri, L. Mus, F. Blandini, Parkinson's Disease in Women and Men: What's the Difference? J. Parkinsons Dis. 9, 501-515 (2019); S. K. Van Den Eeden et al., Incidence of Parkinson's disease: variation by age, gender, and race/ethnicity. Am. J. Epidemiol. 157, 1015-1022 (2003)), with men exhibiting worse executive function (T. H. Reekes et al., Sex specific cognitive differences in Parkinson disease. NPJ Parkinsons Dis. 6, 7 (2020)), while women have a greater risk of disability from therapy related complications. M. Picillo et al., The relevance of gender in Parkinson's disease: a review. J. Neurol. 264, 1583-1607 (2017).

Toxin exposure: Adult common marmosets (Callithrix jacchus, N=22; ages 3-15 years) were housed at the Southwest National Primate Research Center (Texas Biomedical Research Institute, USA) and fed diets supplemented with β-N-methylamino-L-alanine [L-BMAA-HCl, 154.6 MW, proprietary synthesis with L-optical rotation and 98.17% purity from Irvine Chemistry Lab, Anaheim CA, was determined to conform to structure by two laboratories and tested against an authenticated BMAA standard produced by Sigma-Aldrich (B-107) according to 1H NMR, 13C NMR, MS, MS/MS, HPLC (Error! Bookmark not defined.)]. L-BMAA alone, L-BMAA+L-serine, or L-BMAA+L-tyrosine continuously in food for 140 days; all compounds were added to the diet at 50 mg/day. Amino acids L-serine and L-tyrosine were obtained from Bulk Supplements, Henderson, Nevada. In Experiment 1 marmosets were fed 50 mg/day L-BMAA-HCl powder (N=4) to compare neuropathology with the control group (N=2). In Experiment 2, marmosets were placed into four treatment groups, each with two males and two females and dosed with L-BMAA-HCl alone, or L-BMAA-HCl in combination with L-serine or L-tyrosine in a 1:1 dose, or control. For experiment 2, 87 grams of L-BMAA-HCl were dissolved into 1.74 liters of 2 mM HCl for liquid dosing (1 ml/day). Body weight measurements were taken before during and after exposure in standard intervals. Dosing based on median body weights at the beginning and end of experiment 2 were as follows: 102 mg/kg/day and 122 mg/kg/day, respectively. We note that because L-BMAA-HCl is a salt the effective dose of L-BMAA was lower representing 38 mg/day. All study procedures were reviewed and approved by the Texas Biomedical Research Institute, Institutional Animal Care and Use Committee (IACUC), and all experiments were performed in accordance with guidelines and regulations. Animals were monitored by Texas Biomedical Research Institute veterinarians and all procedures were consistent with the commonly accepted norms of veterinary best practice. Euthanasia agents were ketamine (10 mg/kg, Im) and pentobarbital (50-100 mg, IP, IV, or intercardiac) performed by an Institutional veterinarian.

Cognitive testing: Prefrontal dysfunction and executive function were evaluated using a detour reach cognitive assessment. C. Kabadayi, K. Bobrowicz, M. Osvath, The detour paradigm in animal cognition. Anim. Cogn. 21, 21-35 (2018). Marmosets were presented with a transparent 5-sided box containing a preferred reward. Animals were initially habituated with the open side of the box facing the subject allowing them to simply reach straight into the box to retrieve the reward. During testing, the box was randomly rotated such that the opening of the box was in a different orientation each trial in a positional pattern according to a random number generator. Individuals were presented with 16-20 trials in each daily session, with a maximum of 30 secs to retrieve the reward. Trials were scored as successful or unsuccessful, and overall % correct was summed for each session. All trials were videotaped and scored by an individual who was blinded to subject treatment. These cognitive tests correlate with alpha-synuclein misfolds, and the inventors discovered that improvements in cognitive functions determined in these testing protocols correlate with reduction in alpha-synuclein misfolds in the L-tyrosine and L-serine cohorts which were co-administered with the neurotoxin BMAA, when compared to the marmoset cohort which solely received the neurotoxin BMAA. This powerful correlation shows that reduction in alpha-synuclein misfolds directly leads to a profound reduction in symptoms of alpha-synuclein misfold diseases

Brain and Spinal Cord Collection: Following chronic dietary exposure, marmosets were euthanized under veterinary care and tissues were rapidly procured. In brief, whole fresh brain was removed from the calvarium of the marmoset, examined, weighed on a digital scale, and washed in buffered saline (pH 6.5-7.0) (VWR™ Life Science; Randor, PA, USA). After rinsing, brains were gently dried with surgical gauze and placed onto a clean plexiglass cutting stage. A sagittal cut was made to bisect the brain into left and right hemispheres. The right hemisphere with brainstem and cerebellum attached, was placed immediately into 10% neutral buffered formalin (VWR™ Life Science; Randor, PA, USA) and the time of fixation was recorded. In order to avoid lateral bias, the fixation of every fourth hemisphere was alternated to the left hemisphere. For spinal cord, tissues were rinsed in normal buffered saline and placed into 10% neutral buffered formalin for processing, embedding, and sectioning.

Immunohistochemistry: Following fixation, brain and spinal cord tissues were processed, embedded, and sectioned into 40 μm sections on 76×51 mm glass slides at NeuroScience Associates (NSA Labs®; Knoxville, TN, USA) using MultiBrain® and MutiCord® Technologies. Brain hemispheres (N=22 marmosets) were randomized per treatment group and 6 hemispheres were embedded per each of the three MultiBrain® blocks. Each MultiBrain® block contained at least one marmoset from each of the four treatment conditions. All spinal cord tissues (n=16 marmosets) were embedded into one MultiCord® block. Spinal cords were only evaluated in the second experiment. After sectioning, antigen retrieval was performed for a complete series spanning the entire brain or spinal cord (every 24th section) were immunostained with mouse phospho-tau Ser202, Thr205 (AT8, 1:50; Thermo Fischer Scientific, Waltham, MA, USA, cat no. MN1020), rabbit anti-alpha-synuclein pS129 (ASYN p^{S129}; 1:1,500; Abcam, Waltham, MA, USA, cat no. ab51253), and rabbit recombinant monoclonal anti-alpha-synuclein aggregate antibody [MJFR-14-6-4-2, 1:500,000; Abcam, Waltham, MA, USA, cat no. ab209538]. Secondary antibodies applied were goat anti-rabbit or goat anti-mouse biotinylated (Vector Laboratories, Newark, CA, USA, cat no. ba-1000 and ba-9200). Nickel enhanced 3,3′-diaminobenzidine (NiDAB) was used as a chromogen. Cellular architecture and nuclei tissue sections were stained with hematoxylin and eosin or a neutral red counterstain. Archived postmortem human brain tissues at NSA were used as positive controls for immunostaining.

Quantitative Pathology: Scans of pS129 α-syn and Tau AT8 immunostained tissue sections were generated using a Digital Tissue Scope™ LE (Huron Digital Pathology, ON, CAN) at 20× & 40× magnification. The following anatomical areas were examined in the marmoset brain: spinal cord, midbrain/brainstem, limbic system, and neocortex. H. Braak et al., Staging of brain pathology related to sporadic Parkinson's disease. Neurobiol. Aging 24, 197-211 (2003). Within these four anatomical areas, pS129 α-syn immunopositive staining was quantified in 16 brain regions: basolateral dorsal amygdaloid nucleus (BLD), paralaminar amygdaloid nucleus (PaL), anterior entorhinal cortex (aEnt), posterior entorhinal cortex (pEnt), frontal cortex area 8 (A8), frontal cortex area 11 (A11), hippocampus (CA3), medulla adjacent to the 4th ventricle (med 4V), medial longitudinal fasciculus (mlf), parietal area (PE), perirhinal area 36 (A36), substantia nigra (Sn), primary visual cortex (V1) & visual area 2 (V2), anterior horn of spinal cord (AntH), and posterior horn of spinal cord (PosH). Brain abbreviations cited here are from the Marmoset Brain in Stereotaxic Coordinates. G. Paxinos, C. Watson, M. Rosa, M. Petrides, H. Tokuno, The Marmoset Brain in Stereotaxic Coordinates, Elsevier Academic Press (2012).

Treatment groups were blinded to the investigator before selection of regions of interest (ROI). For each ROI, a field of interest (1216×686 pixels; RGB color 3.2 MB) was selected from each marmoset using ObjectiveView™ Digital Pathology Image Viewer (Objective Pathology Services ON, CAN) and then exported into FIJI ImageJ2 software Ver2.0.0-rc-69/1.2p (J. Schindelin et al., Fiji: an open-source platform for biological-image analysis. Nat. Methods 9, 676-682 (2012)). The FIJI file was then imported into QuPath Ver 0.3.2 (P. Bankhead et al., QuPath: Open source software for digital pathology image analysis. Sci. Rep. 7, 16878 (2017) for automated image analyses of immunopositive staining using a 2×5 grid yielding 10 analysis fields each at 200 m². Enumeration of immunostaining used the Positive cell detection function for optical density sum of pS129 α-syn and Hematoxylin OD for Tau AT8.

The intensity threshold parameters for pS129 α-syn were as follows: Cell detection: (DAB OD mean) used a nucleus threshold background radius (8 px and an overall intensity threshold of 0.1. The intensity threshold parameters for Tau AT8 were identical except for Threshold 1+ (0.3). For aggregate analysis of pS129 α-syn, cell detection: (DAB OD sum) used a nucleus threshold background radius (1 px) and an overall intensity threshold of 0.1. When performing quantitative analyses on scanned slides, the treatment conditions were blinded to the analyst. For the first experiment: Marmosets (N=6) were divided into 2 groups based on their treatment condition with each group contained equal distribution of males and females. For the second experiment, Marmosets (N=16) were divided into 4 groups based on their treatment condition. Each group contained 4 marmosets (2 male and 2 female). Validation of the quantitative analysis can be seen in the supplemental materials (FIG. 5).

### Statistical Analysis

α-synuclein Aggregates as a Function of Treatment Group and Sex

Given the two-way factorial structure of the experiment with treatment group and sex as the primary factors of interest, the α-synuclein aggregates were analyzed using a two-way ANOVA using the Im( ) function in R{Team, 2023 #41}. The ANOVA of α-synuclein positive staining aggregates by group and sex are shown in Table 6 below:

TABLE 6
ANOVA of α-synuclein positive
staining aggregates by group and sex

| | | Sum | Mean | F | p- | Perm | Partial |
|---|---|---|---|---|---|---|---|
| | Df | Sq | Sq | value | Value | P | η² |
| Group | 3 | 410.6 | 136.85 | 10.303 | 0.00402 | 0.0039 | 0.500 |
| Sex | 1 | 267.0 | 267.3 | 20.104 | 0.00205 | 0.0014 | 0.325 |
| group:Sex | 3 | 36.7 | 12.25 | 0.922 | 0.47294 | 0.4671 | 0.45 |
| Residuals | 8 | 106.3 | 13.28 | | | | |

Three null hypotheses were considered: (i) the effects for the groups are equal, (ii) the effects for males and females are equal, and (iii) there is no interaction between group and sex. Each of these hypotheses was evaluated using the F-distribution-based p-values obtained from the F statistic associated with each of the three null hypotheses. Tukey-adjusted comparisons were calculated for all pairwise differences in group means. For each main effect in the model, a permutation test p-value for each F statistic which does not depend upon assumptions of normality for the data was also used. In all cases, the differences in p-values arising from F tests and permutation tests were negligible, so p-values from the F tests were reported.

### Association Between α-Synuclein Aggregates and Paired Helical Filament Tau Aggregates

We quantify the relationship between α-synuclein aggregates and paired helical filament tau aggregates using Spearman rank correlation (and associated p-values) due to the right-skewness of each variable. Calculations were made using cor.test ( ) in R.

### Paired Helical Filament Tau Aggregates as a Function of Treatment Group and Sex

The statistical analysis plan for evaluating impacts on tau aggregates was identical to the analysis plan for α-synuclein aggregates as described above.

### DRT Scores as a Function of Treatment Group and Sex

Due to skewness in the DRT scores (quantified as sessions to complete the task), all analyses were carried out on the logarithm of the DRT score. The statistical analysis plan for evaluating impacts on log (DRT score) was identical to the analysis plan for α-synuclein aggregates as described above.

### Additional Analyses

Additional statistical analyses (Wilcoxon test, Friedman test with Dunn's multiple comparisons test and Spearman r) were performed using Prism Ver 9.5.1 (Graph Pad, USA).

### Specific Embodiments

L-DOPA is used in the treatment of Parkinson's disease, but prolonged treatment does not slow α-synucleinopathy or cognitive decline. The dietary amino acid L-tyrosine, a biosynthetic precursor to dopamine, has been considered safe in clinical trials but in the 1970s was rejected based on theoretical considerations as a possible treatment for Parkinson's disease. We show that L-tyrosine, when administered in a non-human primate model of α-synucleinopathy, reduces aggregated «-synuclein and protects against cognitive decline. Our results suggest L-tyrosine should be further evaluated as a potential therapeutic molecule for-synucleinopathies.

One embodiment includes a method of treating α-synucleinopathies in a mammal comprising administering the mammal a therapeutically effective amount of a composition comprising L-tyrosine or a derivative or salt thereof to reduce aggregated α-synuclein. Another embodiment includes a method of treating α-synucleinopathies in a mammal comprising administering the mammal a therapeutically effective amount of a composition comprising L-tyrosine or a derivative or salt thereof, and a composition comprising L-serine or a derivative or salt thereof, to reduce aggregated α-synuclein. In this embodiment, the L-tyrosine and L-serine may be in the same or different compositions. In one embodiment, the amounts of L-tyrosine are within the range of from about 0.1 g to about 100 g per day, or from about 1 g to about 50 g per day, or from about 5 g to about 25 g per day, or from about 10 g to about 25 g per day, or from about 15 g to 25 g per day, or about 25 g per day. In another embodiment in which L-serine is used in combination with L-tyrosine, the L-serine can be used in an amount within the range of from about 0.01 to about 40 g per day, or from about 0.1 g to about 30 g per day, or from about 1 g to about 20 g per day, or from about 2 g to about 15 g per day, or from about 2.5 g to 10 g per day, or about 5 g per day.

Another embodiment may include a method of hindering or preventing α-synucleinopathy misfolds, and/or or to methods of proactively treating individuals at risk of α-synucleinopathy misfolds and associated disease comprising administering the mammal a therapeutically effective amount of a composition comprising L-tyrosine or a derivative or salt thereof to reduce aggregated α-synuclein. Another embodiment includes a method of hindering or preventing α-synucleinopathy misfolds, and/or or to methods of proactively treating individuals at risk of α-synucleinopathy misfolds and associated disease comprising administering the mammal a therapeutically effective amount of a composition comprising L-tyrosine or a derivative or salt thereof, and a composition comprising L-serine or a derivative or salt thereof, to reduce aggregated α-synuclein. In this embodiment, the L-tyrosine and L-serine may be in the same or different compositions. The same respective amounts of L-tyrosine and L-serine may be used, including a combination of both in a total amount of 30 mg/day (i.e., 25 mg/day of L-tyrosine and 5 mg/day L-serine, or 15 mg/day of L-tyrosine and 15 mg/day of L-serine, etc.).

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. Some embodiments of the technology described herein suitably can be practiced in the absence of an element not specifically disclosed herein. Accordingly, in some embodiments the term “comprising” or “comprises” can be replaced with “consisting essentially of” or “consisting of” or grammatical variations thereof. A composition “consisting essentially of” refers to a composition that includes only the active ingredients claimed (e.g., active ingredient (AI) or active pharmaceutical ingredient (API); e.g., L-serine, a salt, metabolic precursor, derivative or conjugate thereof); which composition may include other ingredients such as formulation materials, excipients, additives, carriers, preservatives, diluents, solvents, fillers, salts, buffers, coatings, binders, and lubricating agents; and which composition excludes other APIs not claimed.

The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

### REFERENCES

1. H. Fujiwara et al., alpha-Synuclein is phosphorylated in synucleinopathy lesions. Nat. Cell Biol. 4, 160-164 (2002). https://doi.org/10.1038/ncb748
2. A. L. Mahul-Mellier et al., The process of Lewy body formation, rather than simply alpha-synuclein fibrillization, is one of the major drivers of neurodegeneration. Proc. Natl. Acad. Sci. U.S.A. 117, 4971-4982 (2020). https://doi.org/10.1073/pnas.1913904117
3. C. W. Shults, Lewy bodies. Proc. Natl. Acad. Sci. U.S.A. 103, 1661-1668 (2006). https://doi.org/10.1073/pnas.0509567103
4. H. Braak et al., Staging of brain pathology related to sporadic Parkinson's disease. Neurobiol. Aging 24, 197-211 (2003). https://doi.org/10.1016/s0197-4580 (02) 00065-9
5. K. C. Luk et al., Pathological alpha-synuclein transmission initiates Parkinson-like neurodegeneration in nontransgenic mice. Science 338, 949-953 (2012). https://doi.org/0.1126/science.1227157
6. E. Kovari, J. Horvath, C. Bouras, Neuropathology of Lewy body disorders. Brain Res. Bull. 80, 203-210 (2009) https://doi.org/10.1016/j.brainresbull.2009.06.018
7. E. Melamed, F. Hefti, D. J. Pettibone, J. Liebman, R. J. Wurtman, Aromatic L-amino acid decarboxylase in rat corpus striatum: Implications for action of L-dopa in parkinsonism. Neurology, 31, 651-651 (1981). https://doi.org/10.1212/WNL.31.6.651
8. W. Birkmayer, O. Hornykiewicz, The effect of I-3, 4-dihydroxyphenylalanine (=DOPA) on akinesia in parkinsonism. Parkinsonism Relat. Disord. 4, 59-60 (1998). https://doi.org/10.1016/S1353-8020 (98) 00013-3
9. S. W. Chan et al., L-DOPA is incorporated into brain proteins of patients treated for Parkinson's disease, inducing toxicity in human neuroblastoma cells in vitro. Exp. Neurol. 238, 29-37 (2012). https://doi.org/10.1016/j.expneurol.2011.09.029
10. S. Fahn et al., Levodopa and the progression of Parkinson's disease. N. Engl. J. Med. 351, 2498-2508 (2004). https://doi.org/10.1056/NEJMoa033447
11. S. Giannopoulos, K. Samardzic, B. B. A. Raymond, S. P. Djordjevic, K. J. Rodgers, L-DOPA causes mitochondrial dysfunction in vitro: A novel mechanism of L-DOPA toxicity uncovered. Int. J. Biochem. Cell Biol. 117, 105624 (2019). https://doi.org/10.1016/j.biocel.2019.105624
12. G. Fabbrini, J. M. Brotchie, F. Grandas, M. Nomoto, C. G. Goetz, Levodopa-induced dyskinesias. Mov. Disord., 22, 1379-1389 (2007). https://doi.org/10.1002/mds.21475
13. J. H. Growdon, E. Melamed, M. Logue, F. Hefti, R. J. Wurtman, Effects of oral L-tyrosine administration on CSF tyrosine and homovanillic acid levels in patients with Parkinson's disease. Life Sci. 30, 827-832 (1982). https://doi.org/10.1016/0024-3205 (82) 90596-3
14. P. B. Molinoff, J. U. Axelrod, Biochemistry of catecholamines. Annu. Rev. Biochem. 40, 465-500 (1971).
15. A. Abbott, Levodopa: the story so far. Nature 466, S6-7 (2010). https://doi.org/10.1038/466S6a
16. A. J. Montgomery, et al., Reduction of brain dopamine concentration with dietary tyrosine plus phenylalanine depletion: an [11C]raclopride PET study. Am. J. Psychiatry 160, 1887-1889 (2003). https://doi.org/10.1176/appi.ajp.160.10.1887
17. A. McCann et al., Serum tyrosine is associated with better cognition in Lewy body dementia. Brain Res 1765, 147481 (2021). https://doi.org/10.1016/j.brainres.2021.147481
18. V. Jackson-Lewis, S. Przedborski, Protocol for the MPTP mouse model of Parkinson's disease. Nat. Protoc. 2, 141-151 (2007). https://doi.org/10.1038/nprot.2006.342
19. N. W. Kowall et al., MPTP induces alpha-synuclein aggregation in the substantia nigra of baboons. Neuroreport 11, 211-213 (2000). https://doi.org/10.1097/00001756-200001170-00041
20. A. L. McCormack et al., Pathologic modifications of alpha-synuclein in 1-methyl-4-phenyl-1,2,3,6-tetrahydropyridine (MPTP)-treated squirrel monkeys. J. Neuropathol. Exp. Neurol. 67, 793-802 (2008). https://doi.org/10.1097/NEN.0b013e318180f0bd
21. P. A. Cox, D. A. Davis, D. C. Mash, J. S. Metcalf, S. A. Banack, Dietary exposure to an environmental toxin triggers neurofibrillary tangles and amyloid deposits in the brain. Proc. Biol. Sci. 283 (2016). https://doi.org/10.1098/rspb.2015.2397
22. S. A. Banack, P. A. Cox, Creating a Simian Model of Guam ALS/PDC Which Reflects Chamorro Lifetime BMAA Exposures. Neurotox. Res. 33, 24-32 (2018). https://doi.org/10.1007/s12640-017-9745-9746
23. C. Kabadayi, K. Bobrowicz, M. Osvath, The detour paradigm in animal cognition. Anim. Cogn. 21, 21-35 (2018). https://doi.org/10.1007/s10071-017-1152-0
24. S. S. Ghanem et al., α-Synuclein phosphorylation at serine 129 occurs after initial protein deposition and inhibits seeded fibril formation and toxicity. Proc. Natl. Acad. Sci. U.S.A. 119, e2109617119 (2022). https://doi.org/10.1073/pnas.2109617119
25. D. A. Davis et al., L-serine reduces spinal cord pathology in a vervet model of preclinical ALS/MND. J. Neuropath. Exp. Neurol. 79, 393-406 (2020). https://doi.org/10.1093/jnen/nlaa002
26. I. Alafuzoff et al., Staging/typing of Lewy body related alpha-synuclein pathology: a study of the BrainNet Europe Consortium. Acta Neuropathol. 117, 635-652 (2009). https://doi.org/10.1007/s00401-009-0523-2
27. M. Goedert, et al., 100 years of Lewy pathology. Nature reviews. Neurology 9, 13-24 (2013). https://doi.org/10.1038/nrneurol.2012.242
28. M. Sakamoto et al., Heterogeneity of nigral and cortical Lewy bodies differentiated by amplified triple-labeling for alpha-synuclein, ubiquitin, and thiazin red. Exp. Neurol. 177, 88-94 (2002). https://doi.org/10.1006/exnr.2002.7961
29. K. Del Tredici, H. Braak, Spinal cord lesions in sporadic Parkinson's disease. Acta neuropathologica 124, 643-664 (2012). https://doi.org/10.1007/s00401-012-1028-y
30. P. Calabresi et al., Alpha-synuclein in Parkinson's disease and other synucleinopathies: from overt neurodegeneration back to early synaptic dysfunction. Cell Death Dis. 14, 176 (2023). https://doi.org/10.1038/s41419-023-05672-9
31. R. G. Perez et al., A role for alpha-synuclein in the regulation of dopamine biosynthesis. J. Neurosci. 22, 3090-3099 (2002). https://doi.org/10.1523/JNEUROSCI.22-08-03090.2002
32. M. Goedert, et al., Monoclonal antibody AT8 recognises tau protein phosphorylated at both serine 202 and threonine 205. Neurosci. Lett. 189, 167-169 (1995). https://doi.org/10.1016/0304-3940 (95) 11484-E
33. S. Moussaud et al., Alpha-synuclein and tau: teammates in neurodegeneration? Mol. Neurodegener. 9, 43 (2014). https://doi.org/10.1186/1750-1326-9-43
34. U. Sengupta et al., Pathological interface between oligomeric alpha-synuclein and tau in synucleinopathies. Biol. Psychiatry 78, 672-683 (2015). https://doi.org/10.1016/j.biopsych.2014.12.019
35. D. O. Astrom et al., High risk of developing dementia in Parkinson's disease: a Swedish registry-based study. Sci. Rep. 12, 16759 (2022). https://doi.org/10.1038/s41598-022-21093-8
36. L. Pan, L. Meng, M. He, Z. Zhang, Tau in the Pathophysiology of Parkinson's Disease. J. Mol. Neurosci. 71, 2179-2191 (2021). https://doi.org/10.1007/s12031-020-01776-5
37. S. Cerri, L. Mus, F. Blandini, Parkinson's Disease in Women and Men: What's the Difference? J. Parkinsons Dis. 9, 501-515 (2019). https://doi.org/10.3233/JPD-191683
38. S. K. Van Den Eeden et al., Incidence of Parkinson's disease: variation by age, gender, and race/ethnicity. Am. J. Epidemiol. 157, 1015-1022 (2003). https://doi.org/10.1093/aje/kwg068
39. T. H. Reekes et al., Sex specific cognitive differences in Parkinson disease. NPJ Parkinsons Dis. 6, 7 (2020). https://doi.org/10.1038/s41531-020-0109-1
40. M. Picillo et al., The relevance of gender in Parkinson's disease: a review. J. Neurol. 264, 1583-1607 (2017). https://doi.org/10.1007/s00415-016-8384-9
41. D. B. Calne, M. Sandler, L-Dopa and parkinsonism. Nature 226, 21-24 (1970). https://doi.org/10.1038/226021a0
42. J. DiFrancisco-Donoghue, E. Rabin, E. M. Lamberg, W. G. Werner, Effects of tyrosine on Parkinson's disease: a randomized, double-blind, placebo-controlled trial. Mov. Disord. Clin. Pract. 1, 348-353 (2014). https://doi.org/10.1002/mdc3.12082
43. L. Steenbergen, et al., Tyrosine promotes cognitive flexibility: evidence from proactive vs. reactive control during task switching performance. Neuropsychologia 69, 50-55 (2015). https://doi.org/10.1016/j.neuropsychologia.2015.01.022
44. M. Bloemendaal et al., Neuro-cognitive effects of acute tyrosine administration on reactive and proactive response inhibition in healthy older adults. Eneuro 5, (2018). https://doi.org/10.1523/ENEURO.0035-17.2018
45. C. Klein, A. Westenberger, Genetics of Parkinson's disease. Cold Spring Harb. Perspect. Med. 2, a008888 (2012). https://doi.org/10.1101/cshperspect.a008888
46. R. Savica, et al., When does Parkinson disease start? Arch. Neurol. 67, 798-801 (2010). https://doi.org/10.1001/archneurol.2010.135
47. D. Saredakis, et al., Conversion to MCI and dementia in Parkinson's disease: a systematic review and meta-analysis. Parkinsonism Relat. Disord. 65, 20-31 (2019). https://doi.org/10.1016/j.parkreldis.2019.04.020
48. G. Paxinos, C. Watson, M. Rosa, M. Petrides, H. Tokuno, The Marmoset Brain in Stereotaxic Coordinates, Elsevier Academic Press (2012).
49. J. Schindelin et al., Fiji: an open-source platform for biological-image analysis. Nat. Methods 9, 676-682 (2012). https://doi.org/10.1038/nmeth.2019
50. P. Bankhead et al., QuPath: Open source software for digital pathology image analysis. Sci. Rep. 7, 16878 (2017). https://doi.org/10.1038/s41598-017-17204-5

What is claimed is:

1. A method of treating α-synucleinopathies in a mammal comprising administering to a mammal in need thereof a therapeutically effective amount of an L-tyrosine compound sufficient to prevent, inhibit or reduce misfoldings or aggregates of α-synuclein in the brain of the mammal.

2. The method of claim 1, wherein the α-synucleinopathies are selected from the group consisting of Parkinson's disease (PD), dementia with Lewy Bodies (DLB), and Multiple system atrophy (MSA).

3. The method of claim 2, wherein the α-synucleinopathy is Parkinson's disease (PD).

4. The method of claim 1, wherein the L-tyrosine compound is selected from the group consisting of an L-tyrosine polymer (polytyrosine), an alkylated L-tyrosine, L-tyrosine having an alkyl group comprising from 1-20 carbon atoms, an L-tyrosine lipid, a salt of L-tyrosine selected from a sodium salt, a potassium salt, a calcium salt, a magnesium salt, a zinc salt, or an ammonium salt, an L-tyrosine ester, an L-tyrosine di-ester, a phosphate ester of L-tyrosine, a sulfate or sulfonate ester of L-tyrosine, a pegylated L-tyrosine, an L-tyrosine with one or more polyethylene glycol (PEG) moieties, L-phosphoserine, and mixtures and combinations thereof.

5. The method of claim 1, wherein the L-tyrosine compound is present in a composition comprising a pharmaceutically acceptable carrier.

6. The method of claim 1, wherein the L-tyrosine compound is administered by a method selected from the group consisting of orally, injection, infusion, intubation, catheter, and intracranially.

7. The method of claim 1, wherein the L-tyrosine compound is administered in an amount of from about 1 g to about 50 g per day.

8. The method of claim 7, wherein the L-tyrosine compound is administered in an amount of about 25 g per day.

9. A method of hindering or preventing α-synucleinopathy misfolds or aggregates, and/or proactively treating individuals at risk of α-synucleinopathy misfolds or aggregates in a mammal comprising administering to the mammal a therapeutically effective amount of an L-tyrosine compound sufficient to prevent, inhibit or reduce misfoldings or aggregates of α-synuclein in the brain of the mammal.

10. The method of claim 9, wherein the α-synucleinopathy misfolds or aggregates cause an α-synucleinopathy selected from the group consisting of Parkinson's disease (PD), dementia with Lewy Bodies (DLB), and Multiple system atrophy (MSA).

11. The method of claim 9, wherein the L-tyrosine compound is selected from the group consisting of an L-tyrosine polymer (polytyrosine), an alkylated L-tyrosine, L-tyrosine having an alkyl group comprising from 1-20 carbon atoms, an L-tyrosine lipid, a salt of L-tyrosine selected from a sodium salt, a potassium salt, a calcium salt, a magnesium salt, a zinc salt, or an ammonium salt, an L-tyrosine ester, an L-tyrosine di-ester, a phosphate ester of L-tyrosine, a sulfate or sulfonate ester of L-tyrosine, a pegylated L-tyrosine, an L-tyrosine with one or more polyethylene glycol (PEG) moieties, L-phosphoserine, and mixtures and combinations thereof.

12. The method of claim 9, wherein the L-tyrosine compound is present in a composition comprising a pharmaceutically acceptable carrier.

13. The method of claim 9, wherein the L-tyrosine compound is administered by a method selected from the group consisting of orally, injection, infusion, intubation, catheter, and intracranially.

14. The method of claim 9, wherein the L-tyrosine compound is administered in an amount of from about 1 g to about 50 g per day.

15. The method of claim 14, wherein the L-tyrosine compound is administered in an amount of about 25 g per day.

16. The method of claim 9, further comprising administering a therapeutically effective amount of L-serine.

17. The method of claim 16, wherein the L-serine and L-tyrosine compound are administered in a total combined amount of about 30 g/day.

18. The method of claim 17, wherein L-serine is administered in an amount of from about 0.1 g/day to about 10 g/day.

19. A composition useful in hindering or preventing α-synucleinopathy misfolds or aggregates, and/or proactively treating individuals at risk of α-synucleinopathy misfolds or aggregates in a mammal comprising a therapeutically effective amount of an L-tyrosine compound and an L-serine compound.
